(19) United States
(12) Patent Application Publication
(10) Pub. No.: US 2021/0109107 A1
(43) Pub. Date: Apr. 15, 2021

(54) METHODS FOR CHARACTERIZING HOST-CELL PROTEINS

(71) Applicant: Regeneron Pharmaceuticals, Inc., Tarrytown, NY (US)

(72) Inventors: Xiaojing Zheng, Croton on the Hudson, NY (US); Reid O'Brien Johnson, Hartsdale, NY (US); Tyler Greer, Elmsford, NY (US)

(21) Appl. No.: 17/071,399

(22) Filed: Oct. 15, 2020

Related U.S. Application Data
(60) Provisional application No. 62/915,344, filed on Oct. 15, 2019.
(60) Provisional application No. 62/986,324, filed on Mar. 6, 2020.

(51) Int. Cl. G01N 33/68; G01N 33/94

(52) U.S. Cl. CPC G01N 33/6848; G01N 33/94

(57) ABSTRACT

Methods for characterizing host-cell proteins in a sample matrix are provided.

### FIELD

The present invention generally pertains to characterizing host-cell proteins.

### BACKGROUND

Protein-based biopharmaceutical products have emerged as important drugs for the treatment of cancer, autoimmune disease, infection and cardiometabolic disorders, and they represent one of the fastest growing product segments of the pharmaceutical industry. Bringing a protein-based biotherapeutic to the clinic can be a multiyear undertaking requiring coordinated efforts throughout various research and development disciplines, including discovery, process and formulation development, analytical characterization, and pre-clinical toxicology and pharmacology. Protein-based biopharmaceutical products must meet very high standards of purity. Thus, it can be important to monitor any impurities in such biopharmaceutical products at different stages of drug development, production, storage and handling.

For example, host cell proteins (HCPs) can be present in protein-based biopharmaceuticals which are developed using cell-based systems. The presence of HCPs in drug products needs to be monitored and can be unacceptable above a certain amount. Analytical methods for assays for characterization of HCPs should display sufficient accuracy and resolution. Direct analysis can require isolation of the product in a sufficiently large amount for the assay, which is undesirable and has only been possible in selected cases. Hence, it is a challenging task to determine the workflow and analytical tests to characterize HCPs in a sample matrix when mixed with overwhelmingly high concentration of an active drug. From the foregoing it will be appreciated that a need exists for improved methods for characterizing and monitoring HCPs at various stages of a biopharmaceutical process.

### SUMMARY

A key criterion in developing biopharmaceutical products can be to monitor impurities in the product. When such impurities do occur, their characterization constitutes an important step in the bioprocess.

Exemplary embodiments disclosed herein satisfy the aforementioned demands by providing methods for characterizing host-cell protein(s).

In one exemplary embodiment, the method for characterizing host-cell proteins in a sample matrix can comprise an enrichment step on host-cell proteins in the sample matrix by contacting the sample matrix with a chromatography support and performing a fractionation step. In one aspect, the chromatography support can be an affinity chromatography support. In a specific aspect, the affinity chromatography support can be a protein A chromatography support. In one aspect, the chromatography support can comprise protein A or protein G. In a specific aspect, the protein A or the protein G can be immobilized on agarose or sepharose resin.

In one aspect, the enrichment step can further comprise washing the chromatography support with a wash buffer and collecting a flow-through. In another aspect, the enrichment step can further comprise washing the chromatography support with an elution buffer and collecting the eluted fractions.

In one aspect, the enrichment step can further comprise treating a sample obtained from the chromatography support. In one aspect, the treatment can include adding a hydrolyzing agent to the sample to produce peptides. In one aspect, the treatment can include adding a reducing agent to the sample. In one aspect, the treatment can include adding an alkylating agent to the sample. In another aspect, the treatment can include adding one or more form the group consisting of alkylating agent, reducing agent, hydrolyzing agent or combinations thereof. The additions of these agents to the sample can vary. The addition can be carried by adding the sample to the agents or by adding the agents to the samples.

In another aspect, the sample matrix can further comprise a protein of interest. In a specific aspect, the protein of interest can be an antibody, a bispecific antibody, a multi-specific antibody, an antibody fragment, a monoclonal antibody, a fusion protein, or combinations thereof.

In one aspect, the fractionation step can be a size-based fractionation, a hydrophobicity-based fractionation, a charge-based fractionation, a pI-based fractionation, fractionation by liquid chromatography, or combinations thereof. In a specific aspect, the fractionation step by liquid chromatography can be carried out using reversed phase liquid chromatography.

In another aspect, the method can be capable of characterizing at least about 50% more host-cell proteins than a method comprising the enrichment step and not the fractionation step.

In yet another aspect, the method can be capable of characterizing at least about 50% more host-cell proteins than a method comprising the fractionation step and not the enrichment step.

In one aspect, the method can be capable of characterizing at least about 50% to about 75% more host-cell proteins than a method comprising the enrichment step and not the fractionation step.

In another aspect, the method can be capable of characterizing at least about 50% to about 75% more host-cell proteins than a method comprising the fractionation step and not the enrichment step.

In yet another aspect, the method can further comprise characterizing at least one of the host-cell proteins using a mass spectrometer.

In one exemplary embodiment, the method for characterizing host-cell proteins in a sample matrix can comprise an enrichment step on host-cell proteins in the sample matrix by contacting the sample matrix with an affinity chromatography support and performing a fractionation step. In one aspect, the affinity chromatography support can be a protein A chromatography support. In another aspect, the affinity chromatography support can comprise protein A or protein G. In a specific aspect, the protein A or the protein G can be immobilized on agarose or sepharose resin.

In one aspect, the enrichment step can further comprise washing the chromatography support with a wash buffer and collecting a flow-through. In another aspect, the enrichment step can further comprise washing the chromatography support with an elution buffer and collecting the eluted fractions.

In another aspect, the enrichment step can further comprise treating a sample obtained from the chromatography support. In one aspect, the treatment can include adding a hydrolyzing agent to the sample to produce peptides. In one aspect, the treatment can include adding a reducing agent to the sample. In one aspect, the treatment can include adding an alkylating agent to the sample. In another aspect, the treatment can include adding one or more from the group consisting of alkylating agent, reducing agent, hydrolyzing agent or combinations thereof. The additions of these agents to the sample can vary. The addition can be carried by adding the sample to the agents or by adding the agents to the samples.

In one aspect, the sample matrix can further comprise a protein of interest. In a specific aspect, the protein of interest can be an antibody, a bispecific antibody, a multi-specific antibody, an antibody fragment, a monoclonal antibody, a fusion protein, or combinations thereof.

In another aspect, the fractionation step can be a size-based fractionation, a hydrophobicity-based fractionation, a charge-based fractionation, a pI-based fractionation, fractionation by liquid chromatography, or combinations thereof. In a specific aspect, the fractionation step by liquid chromatography can be carried out using reversed phase liquid chromatography.

In yet another aspect, the method can be capable of characterizing at least about 50% more host-cell proteins than a method comprising the enrichment step and not the fractionation step.

In another aspect, the method can be capable of characterizing at least about 50% more host-cell proteins than a method comprising the fractionation step and not the enrichment step.

In one aspect, the method can be capable of characterizing at least about 50% to about 75% more host-cell proteins than a method comprising the enrichment step and not the fractionation step.

In another aspect, the method can be capable of characterizing at least about 50% to about 75% more host-cell proteins than a method comprising the fractionation step and not the enrichment step.

In one aspect, the method can further comprise characterizing at least one of the host-cell proteins using a mass spectrometer.

In one exemplary embodiment, the method for characterizing host-cell proteins in a sample matrix can comprise an enrichment step on host-cell proteins in the sample matrix by contacting the sample matrix with a chromatography support, performing a fractionation step and characterizing at least one of the host-cell proteins using a mass spectrometer. In one aspect, the chromatography support can be an affinity chromatography support. In a specific aspect, the affinity chromatography support can be a protein A chromatography support. In one aspect, the chromatography support can comprise protein A or protein G. In a specific aspect, the protein A or the protein G can be immobilized on agarose or sepharose resin.

In one aspect, the enrichment step can further comprise washing the chromatography support with a wash buffer and collecting a flow-through. In another aspect, the enrichment step can further comprise washing the chromatography support with an elution buffer and collecting the eluted fractions.

In another aspect, the enrichment step can further comprise treating a sample obtained from the chromatography support. In one aspect, the treatment can include adding a hydrolyzing agent to the sample to produce peptides. In one aspect, the treatment can include adding a reducing agent to the sample. In one aspect, the treatment can include adding an alkylating agent to the sample. In another aspect, the treatment can include adding one or more form the group consisting of alkylating agent, reducing agent, hydrolyzing agent or combinations thereof. The additions of these agents to the sample can vary. The addition can be carried by adding the sample to the agents or by adding the agents to the samples.

In another aspect, the sample matrix can further comprise a protein of interest. In a specific aspect, the protein of interest can be an antibody, a bispecific antibody, a multi-specific antibody, an antibody fragment, a monoclonal antibody, a fusion protein, or combinations thereof.

In yet another aspect, the fractionation step can be a size-based fractionation, a hydrophobicity-based fractionation, a charge-based fractionation, a pI-based fractionation, fractionation by liquid chromatography, or combinations thereof. In a specific aspect, the fractionation step by liquid chromatography can be carried out using reversed phase liquid chromatography.

In one aspect, the method can be capable of characterizing at least about 50% more host-cell proteins than a method comprising the enrichment step and not the fractionation step.

In another aspect, the method can be capable of characterizing at least about 50% more host-cell proteins than a method comprising the fractionation step and not the enrichment step.

In one aspect, the method can be capable of characterizing at least about 50% to about 75% more host-cell proteins than a method comprising the enrichment step and not the fractionation step.

In another aspect, the method can be capable of characterizing at least about 50% to about 75% more host-cell proteins than a method comprising the fractionation step and not the enrichment step.

In yet another aspect, the mass spectrometer can be a tandem mass spectrometer. In another aspect, the mass spectrometer can be coupled with a liquid chromatography system. In an aspect therein, the liquid chromatography system can be a nano-liquid chromatography system. In yet another aspect, the mass spectrometer can be a tandem mass spectrometer coupled with a liquid chromatography system.

In one exemplary embodiment, the method for characterizing host-cell proteins in a sample matrix can comprise an enrichment step on host-cell proteins in the sample matrix by contacting the sample matrix with an affinity chromatography support, performing a fractionation step and characterizing at least one of the host-cell proteins using a mass spectrometer. In a specific aspect, the affinity chromatography support can be a protein A chromatography support. In one aspect, the affinity chromatography support can comprise protein A or protein G. In a specific aspect, the protein A or the protein G can be immobilized on agarose or sepharose resin.

In one aspect, the enrichment step can further comprise washing the chromatography support with a wash buffer and collecting the flow-through. In another aspect, the enrichment step can further comprise washing the chromatography support with an elution buffer and collecting the eluted fractions.

In another aspect, the enrichment step can further comprise treating a sample obtained from the chromatography support. In one aspect, the treatment can include adding a hydrolyzing agent to the sample to produce peptides. In one aspect, the treatment can include adding a reducing agent to the sample. In one aspect, the treatment can include adding an alkylating agent to the sample. In another aspect, the treatment can include adding one or more form the group consisting of alkylating agent, reducing agent, hydrolyzing agent or combinations thereof. The additions of these agents to the sample can vary. The addition can be carried by adding the sample to the agents or by adding the agents to the samples.

In yet another aspect, the sample matrix can further comprise a protein of interest. In a specific aspect, the protein of interest can be an antibody, a bispecific antibody, a multi-specific antibody, an antibody fragment, a monoclonal antibody, a fusion protein, or combinations thereof.

In one aspect, the fractionation step can be a size-based fractionation, a hydrophobicity-based fractionation, a charge-based fractionation, a pI-based fractionation, fractionation by liquid chromatography, or combinations thereof. In a specific aspect, the fractionation step by liquid chromatography can be carried out using reversed phase liquid chromatography.

In one aspect, the method can be capable of characterizing at least about 50% more host-cell proteins than a method comprising the enrichment step and not the fractionation step.

In another aspect, the method can be capable of characterizing at least about 50% more host-cell proteins than a method comprising the fractionation step and not the enrichment step.

In another aspect, the method can be capable of characterizing at least about 50% to about 75% more host-cell proteins than a method comprising the enrichment step and not the fractionation step.

In yet another aspect, the method can be capable of characterizing at least about 50% to about 75% more host-cell proteins than a method comprising the fractionation step and not the enrichment step.

In one aspect, the mass spectrometer can be a tandem mass spectrometer. In another aspect, the mass spectrometer can be coupled with a liquid chromatography system. In an aspect therein, liquid chromatography system can be a nano-liquid chromatography system. In yet another aspect, the mass spectrometer can be a tandem mass spectrometer coupled with a liquid chromatography system.

In one exemplary embodiment, the method for characterizing host-cell proteins in a sample matrix can comprise an enrichment step on host-cell proteins in the sample matrix by contacting the sample matrix with a chromatography support, performing a fractionation step and characterizing at least one of the host-cell proteins using High-Field Asymmetric Waveform Ion Mobility Spectrometry. In one aspect, the chromatography support can be an affinity chromatography support. In a specific aspect, the affinity chromatography support can be a protein A chromatography support. In one aspect, the chromatography support can comprise protein A or protein G. In a specific aspect, the protein A or the protein G can be immobilized on agarose or sepharose resin.

In one aspect, the enrichment step can further comprise washing the chromatography support with a wash buffer and collecting a flow-through. In another aspect, the enrichment step can further comprise washing the chromatography support with an elution buffer and collecting the eluted fractions.

In another aspect, the enrichment step can further comprise treating a sample obtained from the chromatography support. In one aspect, the treatment can include adding a hydrolyzing agent to the sample to produce peptides. In one aspect, the treatment can include adding a reducing agent to the sample. In one aspect, the treatment can include adding an alkylating agent to the sample. In another aspect, the treatment can include adding one or more form the group consisting of alkylating agent, reducing agent, hydrolyzing agent or combinations thereof. The additions of these agents to the sample can vary. The addition can be carried by adding the sample to the agents or by adding the agents to the samples.

In yet another aspect, the sample matrix can further comprise a protein of interest. In a specific aspect, the protein of interest can be an antibody, a bispecific antibody, a multi-specific antibody, an antibody fragment, a monoclonal antibody, a fusion protein, or combinations thereof.

In one aspect, the fractionation step can be a size-based fractionation, a hydrophobicity-based fractionation, a charge-based fractionation, a pI-based fractionation, fractionation by liquid chromatography, or combinations thereof. In a specific aspect, the fractionation step by liquid chromatography can be carried out using reversed phase liquid chromatography.

In another aspect, the method can be capable of characterizing at least about 50% more host-cell proteins than a method comprising the enrichment step and not the fractionation step.

In yet another aspect, the method can be capable of characterizing at least about 50% more host-cell proteins than a method comprising the fractionation step and not the enrichment step.

In one aspect, the method can be capable of characterizing at least about 50% to about 75% more host-cell proteins than a method comprising the enrichment step and not the fractionation step.

In another aspect, the method can be capable of characterizing at least about 50% to about 75% more host-cell proteins than a method comprising the fractionation step and not the enrichment step.

In one exemplary embodiment, the method for characterizing host-cell proteins in a sample matrix can comprise an enrichment step on host-cell proteins in the sample matrix by contacting the sample matrix with an affinity chromatography support, performing a fractionation step and characterizing at least one of the host-cell proteins using a High-Field Asymmetric Waveform Ion Mobility Spectrometry. In a specific aspect, the affinity chromatography support can be a protein A chromatography support. In one aspect, the affinity chromatography support can comprise protein A or protein G. In a specific aspect, the protein A or the protein G can be immobilized on agarose or sepharose resin.

In one aspect, the enrichment step can further comprise washing the chromatography support with a wash buffer and collecting a flow-through. In another aspect, the enrichment step can further comprise washing the chromatography support with an elution buffer and collecting the eluted fractions.

In one aspect, the enrichment step can further comprise treating a sample obtained from the chromatography support. In one aspect, the treatment can include adding a hydrolyzing agent to the sample to produce peptides. In one aspect, the treatment can include adding a reducing agent to the sample. In one aspect, the treatment can include adding an alkylating agent to the sample. In another aspect, the treatment can include adding one or more form the group consisting of alkylating agent, reducing agent, hydrolyzing agent or combinations thereof. The additions of these agents to the sample can vary. The addition can be carried by adding the sample to the agents or by adding the agents to the samples.

In another aspect, the sample matrix can further comprise a protein of interest. In a specific aspect, the protein of interest can be an antibody, a bispecific antibody, a multi-specific antibody, an antibody fragment, a monoclonal antibody, a fusion protein, or combinations thereof.

In another aspect, the fractionation step can be a size-based fractionation, a hydrophobicity-based fractionation, a charge-based fractionation, a pI-based fractionation, fractionation by liquid chromatography, or combinations thereof. In a specific aspect, the fractionation step by liquid chromatography can be carried out using reversed phase liquid chromatography.

In one aspect, the method can be capable of characterizing at least about 50% more host-cell proteins than a method comprising the enrichment step and not the fractionation step.

In another aspect, the method can be capable of characterizing at least about 50% more host-cell proteins than a method comprising the fractionation step and not the enrichment step.

In one aspect, the method can be capable of characterizing at least about 50% to about 75% more host-cell proteins than a method comprising the enrichment step and not the fractionation step.

In another aspect, the method can be capable of characterizing at least about 50% to about 75% more host-cell proteins than a method comprising the fractionation step and not the enrichment step.

In one exemplary embodiment, the method for characterizing host-cell proteins in a sample matrix having a protein of interest can comprise an enrichment step on host-cell proteins in the sample matrix by contacting the sample matrix with an affinity chromatography support, washing the affinity chromatography support with a wash buffer and collecting the flow-through; and performing a fractionation step. In a specific aspect, the affinity chromatography support can be a protein A chromatography support. In one aspect, the affinity chromatography support can comprise protein A or protein G. In a specific aspect, the protein A or the protein G can be immobilized on agarose or sepharose resin.

In one aspect, the enrichment step can further comprise washing the chromatography support with a wash buffer and collecting a flow-through. In another aspect, the enrichment step can further comprise washing the chromatography support with an elution buffer and collecting the eluted fractions.

In another aspect, the enrichment step can further comprise treating a sample obtained from the chromatography support. In one aspect, the treatment can include adding a hydrolyzing agent to the sample to produce peptides. In one aspect, the treatment can include adding a reducing agent to the sample. In one aspect, the treatment can include adding an alkylating agent to the sample. In another aspect, the treatment can include adding one or more form the group consisting of alkylating agent, reducing agent, hydrolyzing agent or combinations thereof. The additions of these agents to the sample can vary. The addition can be carried by adding the sample to the agents or by adding the agents to the samples.

In one aspect, the sample matrix can further comprise a protein of interest. In a specific aspect, the protein of interest can be an antibody, a bispecific antibody, a multi-specific antibody, an antibody fragment, a monoclonal antibody, a fusion protein, or combinations thereof.

In one aspect, the fractionation step can be can be a size-based fractionation, a hydrophobicity-based fractionation, a charge-based fractionation, a pI-based fractionation, fractionation by liquid chromatography, or combinations thereof. In a specific aspect, the fractionation step by liquid chromatography can be carried out using reversed phase liquid chromatography.

In another aspect, the method can be capable of characterizing at least about 50% more host-cell proteins than a method comprising the enrichment step and not the fractionation step.

In another aspect, the method can be capable of characterizing at least about 50% more host-cell proteins than a method comprising the fractionation step and not the enrichment step.

In one aspect, the method can be capable of characterizing at least about 50% to about 75% more host-cell proteins than a method comprising the enrichment step and not the fractionation step.

In another aspect, the method can be capable of characterizing at least about 50% to about 75% more host-cell proteins than a method comprising the fractionation step and not the enrichment step.

In one aspect, the flow-through can have a reduced amount of protein of interest than the sample matrix.

In another aspect, the method can further comprise characterizing at least one of the host cell proteins using a mass spectrometer. In a specific aspect, the mass spectrometer can be a tandem mass spectrometer. In another specific aspect, the mass spectrometer can be coupled with a liquid chromatography system. In an aspect therein, the liquid chromatography system can be a nano-liquid chromatography system. In yet another specific aspect, the mass spectrometer can be a tandem mass spectrometer coupled with a liquid chromatography system. In another aspect, the method can further comprise characterizing at least one of the host cell proteins using a High-Field Asymmetric Waveform Ion Mobility Spectrometry (FAIMS) device. In another aspect, the method can further comprise characterizing at least one of the host cell proteins using a FAIMS-MS. In another specific aspect, the method can further comprise characterizing at least one of the host cell proteins using FAIMS device in conjunction with LC and MS.

In one exemplary embodiment, the method for characterizing host-cell proteins in a sample matrix can comprise (a) subjecting the sample matrix having host-cell proteins to non-denaturing digestion conditions to form a mixture; and (b) enriching host-cell proteins in said mixture by contacting the mixture with a chromatography support. In one aspect, the chromatography support can be an affinity chromatography support. In a specific aspect, the affinity chromatography support can be a protein A chromatography support. In one aspect, the chromatography support can comprise protein A or protein G. In a specific aspect, the protein A or the protein G can be immobilized on agarose or sepharose resin.

In one aspect, the enrichment step can further comprise washing the chromatography support with a wash buffer and collecting a flow-through. In another aspect, the enrichment step can further comprise washing the chromatography support with an elution buffer and collecting the eluted fractions.

In another aspect, the enrichment step can further comprise treating a sample obtained from the chromatography support. In one aspect, the treatment can include adding a hydrolyzing agent to the sample to produce peptides. In one aspect, the treatment can include adding a reducing agent to the sample. In one aspect, the treatment can include adding an alkylating agent to the sample. In another aspect, the treatment can include adding one or more form the group consisting of alkylating agent, reducing agent, hydrolyzing agent or combinations thereof. The additions of these agents to the sample can vary. The addition can be carried by adding the sample to the agents or by adding the agents to the samples.

In one aspect, the sample matrix can further comprise a protein of interest. In a specific aspect, the protein of interest can be an antibody, a bispecific antibody, a multi-specific antibody, an antibody fragment, a monoclonal antibody, a fusion protein, or combinations thereof.

In another aspect, the method can further comprise characterizing at least one of the host cell proteins using a mass spectrometer. In a specific aspect, the mass spectrometer can be a tandem mass spectrometer. In another specific aspect, the mass spectrometer can be coupled with a liquid chromatography system. In an aspect therein, liquid chromatography system can be a nano-liquid chromatography system. In yet another specific aspect, the mass spectrometer can be a tandem mass spectrometer coupled with a liquid chromatography system. In another aspect, the method can further comprise characterizing at least one of the host cell proteins using a High-Field Asymmetric Waveform Ion Mobility Spectrometry. In another aspect, the method can further comprise characterizing at least one of the host cell proteins using a FAIMS-MS. In another specific aspect, the method can further comprise characterizing at least one of the host cell proteins using FAIMS device in conjunction with LC and MS.

In one aspect, the method can be capable of characterizing at least about 500% more host-cell proteins than a method comprising step (a) and not step (b).

In one aspect, the method can be capable of characterizing at least about 100% to about 1000% more host-cell proteins than a method comprising step (a) and not step (b).

In one exemplary embodiment, the method for characterizing host-cell proteins in a sample matrix can comprise (a) subjecting the sample matrix having host-cell proteins to non-denaturing digestion conditions to form a mixture; and (b) enriching host-cell proteins in said mixture by contacting the mixture with an affinity chromatography support. In one aspect, the affinity chromatography support can be a protein A chromatography support. In one aspect, the chromatography support can comprise protein A or protein G. In a specific aspect, the protein A or the protein G can be immobilized on agarose or sepharose resin.

In one aspect, the enrichment step can further comprise washing the chromatography support with a wash buffer and collecting a flow-through. In another aspect, the enrichment step can further comprise washing the chromatography support with an elution buffer and collecting the eluted fractions.

In another aspect, the enrichment step can further comprise treating a sample obtained from the chromatography support. In one aspect, the treatment can include adding a hydrolyzing agent to the sample to produce peptides. In one aspect, the treatment can include adding a reducing agent to the sample. In one aspect, the treatment can include adding an alkylating agent to the sample. In another aspect, the treatment can include adding one or more form the group consisting of alkylating agent, reducing agent, hydrolyzing agent or combinations thereof. The additions of these agents to the sample can vary. The addition can be carried by adding the sample to the agents or by adding the agents to the samples.

In another aspect, the sample matrix can further comprise a protein of interest. In a specific aspect, the protein of interest can be an antibody, a bispecific antibody, a multi-specific antibody, an antibody fragment, a monoclonal antibody, a fusion protein, or combinations thereof.

In yet another aspect, the method can further comprise characterizing at least one of the host cell proteins using a mass spectrometer. In a specific aspect, the mass spectrometer can be a tandem mass spectrometer. In another specific aspect, the mass spectrometer can be coupled with a liquid chromatography system. In an aspect therein, the liquid chromatography system can be a nano-liquid chromatography system. In yet another specific aspect, the mass spectrometer can be a tandem mass spectrometer coupled with a liquid chromatography system. In another aspect, the method can further comprise characterizing at least one of the host cell proteins using High-Field Asymmetric Waveform Ion Mobility Spectrometry. In another aspect, the method can further comprise characterizing at least one of the host cell proteins using FAIMS-MS. In another specific aspect, the method can further comprise characterizing at least one of the host cell proteins using FAIMS device in conjunction with LC and MS.

In one aspect, the method can be capable of characterizing at least about 500% more host-cell proteins than a method comprising step (a) and not step (b).

In another aspect, the method can be capable of characterizing at least about 100% to about 1000% more host-cell proteins than a method comprising step (a) and not step (b).

In one exemplary embodiment, the method for characterizing host-cell proteins in a sample matrix can comprise (a) subjecting the sample matrix having host-cell proteins to non-denaturing digestion conditions to form a mixture; (b) enriching host-cell proteins in said mixture by contacting the mixture with a chromatography support and (c) characterizing at least one of the host-cell proteins using a mass spectrometer. In one aspect, the chromatography support can be an affinity chromatography support. In a specific aspect, the affinity chromatography support can be a protein A chromatography support. In another specific aspect, the affinity chromatography support can comprise protein A or protein G. In yet another specific aspect, the protein A or the protein G can be immobilized on agarose or sepharose resin.

In one aspect, the enrichment step can further comprise collecting a flow-through from the affinity chromatography support.

In another aspect, the enrichment step can further comprise washing the chromatography support with a wash buffer and collecting the flow-through. In another aspect, the enrichment step can further comprise washing the chromatography support with an elution buffer and collecting the eluted fractions.

In another aspect, the enrichment step can further comprise treating a sample obtained from the chromatography support. In one aspect, the treatment can include adding a hydrolyzing agent to the sample to produce peptides. In one aspect, the treatment can include adding a reducing agent to the sample. In one aspect, the treatment can include adding an alkylating agent to the sample. In another aspect, the treatment can include adding one or more form the group consisting of alkylating agent, reducing agent, hydrolyzing agent or combinations thereof. The additions of these agents to the sample can vary. The addition can be carried by adding the sample to the agents or by adding the agents to the samples.

In one aspect, the sample matrix can further comprise a protein of interest. In a specific aspect, the protein of interest can be an antibody, a bispecific antibody, a multi-specific antibody, an antibody fragment, a monoclonal antibody, a fusion protein, or combinations thereof.

In another aspect, the mass spectrometer can be a tandem mass spectrometer. In another aspect, the mass spectrometer can be coupled with a liquid chromatography system. In an aspect therein, liquid chromatography system can be a nano-liquid chromatography system. In yet another aspect, the mass spectrometer can be a tandem mass spectrometer coupled with a liquid chromatography system.

In yet another aspect, the method can further comprise characterizing at least one of the host cell proteins using High-Field Asymmetric Waveform Ion Mobility Spectrometry. In another aspect, the method can further comprise characterizing at least one of the host cell proteins using FAIMS-MS. In another specific aspect, the method can further comprise characterizing at least one of the host cell proteins using FAIMS device in conjunction with LC and MS.

In one aspect, the method can be capable of characterizing at least about 500% more host-cell proteins than a method comprising step (a) and not step (b).

In another aspect, the method can be capable of characterizing at least about 100% to about 1000% more host-cell proteins than a method comprising step (a) and not step (b).

In one exemplary embodiment, the method for characterizing host-cell proteins in a sample matrix can comprise (a) subjecting the sample matrix having host-cell proteins to non-denaturing digestion conditions to form a mixture; (b) enriching host-cell proteins in said mixture by contacting the mixture with an affinity chromatography support and (c) characterizing at least one of the host-cell proteins using a mass spectrometer. In one aspect, the affinity chromatography support can be a protein A chromatography support. In one aspect, the affinity chromatography support can comprise protein A or protein G. In a specific aspect, the protein A or the protein G can be immobilized on agarose or sepharose resin.

In one aspect, the enrichment step can further comprise washing the chromatography support with a wash buffer and collecting a flow-through. In another aspect, the enrichment step can further comprise washing the chromatography support with an elution buffer and collecting the eluted fractions.

In another aspect, the enrichment step can further comprise treating a sample obtained from the chromatography support. In one aspect, the treatment can include adding a hydrolyzing agent to the sample to produce peptides. In one aspect, the treatment can include adding a reducing agent to the sample. In one aspect, the treatment can include adding an alkylating agent to the sample. In another aspect, the treatment can include adding one or more form the group consisting of alkylating agent, reducing agent, hydrolyzing agent or combinations thereof. The additions of these agents to the sample can vary. The addition can be carried by adding the sample to the agents or by adding the agents to the samples.

In another aspect, the sample matrix can further comprise a protein of interest. In a specific aspect, the protein of interest can be an antibody, a bispecific antibody, a multi-specific antibody, an antibody fragment, a monoclonal antibody, a fusion protein, or combinations thereof.

In yet another aspect, the mass spectrometer can be a tandem mass spectrometer. In another aspect, the mass spectrometer can be coupled with a liquid chromatography system. In an aspect therein, the liquid chromatography system can be a nano-liquid chromatography system. In yet another aspect, the mass spectrometer can be a tandem mass spectrometer coupled with a liquid chromatography system. In another aspect, the method can further comprise characterizing at least one of the host cell proteins using High-Field Asymmetric Waveform Ion Mobility Spectrometry. In another aspect, the method can further comprise characterizing at least one of the host cell proteins using FAIMS-MS. In another specific aspect, the method can further comprise characterizing at least one of the host cell proteins using FAIMS device in conjunction with LC and MS.

In one aspect, the method can be capable of characterizing at least about 500% more host-cell proteins than a method comprising step (a) and not step (b).

In one aspect, the method can be capable of characterizing at least about 100% to about 1000% more host-cell proteins than a method comprising step (a) and not step (b).

In one exemplary embodiment, the method for characterizing host-cell proteins in a sample matrix can comprise enriching host-cell proteins in the sample matrix by contacting the sample matrix with a chromatography support and characterizing at least one of the host-cell proteins using a High-Field Asymmetric Waveform Ion Mobility Spectrometry. In one aspect, the chromatography support can be an affinity chromatography support. In a specific aspect, the affinity chromatography support can be a protein A chromatography support. In one aspect, the chromatography support can comprise protein A or protein G. In a specific aspect, the protein A or the protein G can be immobilized on agarose or sepharose resin.

In one aspect, the enrichment step can further comprise washing the chromatography support with a wash buffer and collecting the flow-through. In another aspect, the enrichment step can further comprise washing the chromatography support with an elution buffer and collecting the eluted fractions.

In another aspect, the enrichment step can further comprise treating a sample obtained from the chromatography support. In one aspect, the treatment can include adding a hydrolyzing agent to the sample to produce peptides. In one aspect, the treatment can include adding a reducing agent to the sample. In one aspect, the treatment can include adding an alkylating agent to the sample. In another aspect, the treatment can include adding one or more form the group consisting of alkylating agent, reducing agent, hydrolyzing agent or combinations thereof. The additions of these agents to the sample can vary. The addition can be carried by adding the sample to the agents or by adding the agents to the samples.

In one aspect, the sample matrix can further comprise a protein of interest. In a specific aspect, the protein of interest can be an antibody, a bispecific antibody, a multi-specific antibody, an antibody fragment, a monoclonal antibody, a fusion protein, or combinations thereof.

In one aspect, the method can be capable of characterizing at least about 30% more host-cell proteins compared to a method not comprising High-Field Asymmetric Waveform Ion Mobility Spectrometry.

In another aspect, the method can be capable of characterizing at least about 30% to about 75% more host-cell proteins compared to a method not comprising High-Field Asymmetric Waveform Ion Mobility Spectrometry.

In one exemplary embodiment, the method for characterizing host-cell proteins in a sample matrix can comprise enriching host-cell proteins in the sample matrix by contacting the sample matrix with an affinity chromatography support and characterizing at least one of the host-cell proteins using a High-Field Asymmetric Waveform Ion Mobility Spectrometry. In one aspect, the affinity chromatography support can be a protein A chromatography support. In one aspect, the affinity chromatography support can comprise protein A or protein G. In a specific aspect, the protein A or the protein G can be immobilized on agarose or sepharose resin.

In one aspect, the enrichment step can further comprise washing the chromatography support with a wash buffer and collecting a flow-through. In another aspect, the enrichment step can further comprise washing the chromatography support with an elution buffer and collecting the eluted fractions.

In another aspect, the enrichment step can further comprise treating a sample obtained from the chromatography support. In one aspect, the treatment can include adding a hydrolyzing agent to the sample to produce peptides. In one aspect, the treatment can include adding a reducing agent to the sample. In one aspect, the treatment can include adding an alkylating agent to the sample. In another aspect, the treatment can include adding one or more form the group consisting of alkylating agent, reducing agent, hydrolyzing agent or combinations thereof. The additions of these agents to the sample can vary. The addition can be carried by adding the sample to the agents or by adding the agents to the samples.

In one aspect, the sample matrix can further comprise a protein of interest. In a specific aspect, the protein of interest can be an antibody, a bispecific antibody, a multi-specific antibody, an antibody fragment, a monoclonal antibody, a fusion protein, or combinations thereof.

In one aspect, the method can be capable of characterizing at least about 30% more host-cell proteins compared to a method not comprising High-Field Asymmetric Waveform Ion Mobility Spectrometry.

In another aspect, the method can be capable of characterizing at least about 30% to about 75% more host-cell proteins compared to a method not comprising High-Field Asymmetric Waveform Ion Mobility Spectrometry.

In one exemplary embodiment, the method for characterizing host-cell proteins in a sample matrix can comprise (a) subjecting the sample matrix having host-cell proteins to non-denaturing digestion conditions to form a mixture, (b) enriching host-cell proteins in said mixture by contacting the mixture with a chromatography support and (c) characterizing of at least one of the host-cell proteins using High-Field Asymmetric Waveform Ion Mobility Spectrometry. In one aspect, the chromatography support can be an affinity chromatography support. In a specific aspect, the affinity chromatography support can be a protein A chromatography support. In one aspect, the chromatography support can comprise protein A or protein G. In a specific aspect, the protein A or the protein G can be immobilized on agarose or sepharose resin.

In one aspect, the enrichment step can further comprise washing the chromatography support with a wash buffer and collecting the flow-through. In another aspect, the enrichment step can further comprise washing the chromatography support with an elution buffer and collecting the eluted fractions.

In another aspect, the enrichment step can further comprise treating a sample obtained from the chromatography support. In one aspect, the treatment can include adding a hydrolyzing agent to the sample to produce peptides. In one aspect, the treatment can include adding a reducing agent to the sample. In one aspect, the treatment can include adding an alkylating agent to the sample. In another aspect, the treatment can include adding one or more form the group consisting of alkylating agent, reducing agent, hydrolyzing agent or combinations thereof. The additions of these agents to the sample can vary. The addition can be carried by adding the sample to the agents or by adding the agents to the samples.

In one aspect, the sample matrix can further comprise a protein of interest. In a specific aspect, the protein of interest can be an antibody, a bispecific antibody, a multi-specific antibody, an antibody fragment, a monoclonal antibody, a fusion protein, or combinations thereof.

In another aspect, the method can further comprise characterizing at least one of the host cell proteins using a mass spectrometer. In a specific aspect, the mass spectrometer can be a tandem mass spectrometer. In another specific aspect, the mass spectrometer can be coupled with a liquid chromatography system. In an aspect therein, the liquid chromatography system can be a nano-liquid chromatography system. In yet another specific aspect, the mass spectrometer can be a tandem mass spectrometer coupled with a liquid chromatography system. In another aspect, the method can further comprise characterizing at least one of the host cell proteins using FAIMS-MS. In another specific aspect, the method can further comprise characterizing at least one of the host cell proteins using FAIMS device in conjunction with LC and MS.

In one aspect, the method can be capable of characterizing at least about 15% more host-cell proteins than a method comprising steps (a) and (b) but not step (c).

In yet another aspect, the method can be capable of characterizing at least about 15% to about 60% more host-cell proteins than a method comprising steps (a) and (b) but not step (c).

In one exemplary embodiment, the method for characterizing host-cell proteins in a sample matrix can comprise (a) subjecting the sample matrix having host-cell proteins to non-denaturing digestion conditions to form a mixture, (b) enriching host-cell proteins in said mixture by contacting the mixture with an affinity chromatography support and (c) characterizing of at least one of the host-cell proteins using High-Field Asymmetric Waveform Ion Mobility Spectrometry. In one aspect, the affinity chromatography support can be a protein A chromatography support. In one aspect, the chromatography support can comprise protein A or protein G. In a specific aspect, the protein A or the protein G can be immobilized on agarose or sepharose resin.

In one aspect, the enrichment step can further comprise washing the chromatography support with a wash buffer and collecting the flow-through. In another aspect, the enrichment step can further comprise washing the chromatography support with an elution buffer and collecting the eluted fractions.

In another aspect, the enrichment step can further comprise treating a sample obtained from the chromatography support. In one aspect, the treatment can include adding a hydrolyzing agent to the sample to produce peptides. In one aspect, the treatment can include adding a reducing agent to the sample. In one aspect, the treatment can include adding an alkylating agent to the sample. In another aspect, the treatment can include adding one or more form the group consisting of alkylating agent, reducing agent, hydrolyzing agent or combinations thereof. The additions of these agents to the sample can vary. The addition can be carried by adding the sample to the agents or by adding the agents to the samples.

In one aspect, the sample matrix can further comprise a protein of interest. In a specific aspect, the protein of interest can be an antibody, a bispecific antibody, a multi-specific antibody, an antibody fragment, a monoclonal antibody, a fusion protein, or combinations thereof.

In another aspect, the method can further comprise characterizing at least one of the host cell proteins using a mass spectrometer. In a specific aspect, the mass spectrometer can be a tandem mass spectrometer. In another specific aspect, the mass spectrometer can be coupled with a liquid chromatography system. In an aspect therein, the liquid chromatography system can be a nano-liquid chromatography system. In yet another specific aspect, the mass spectrometer can be a tandem mass spectrometer coupled with a liquid chromatography system. In another aspect, the method can further comprise characterizing at least one of the host cell proteins using FAIMS-MS. In another specific aspect, the method can further comprise characterizing at least one of the host cell proteins using FAIMS device in conjunction with LC and MS.

In one aspect, the method can be capable of characterizing at least about 15% more host-cell proteins than a method comprising steps (a) and (b) but not step (c).

In yet another aspect, the method can be capable of characterizing at least about 15% to about 60% more host-cell proteins than a method comprising steps (a) and (b) but not step (c).

These, and other, aspects of the invention will be better appreciated and understood when considered in conjunction with the following description and the accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the number of proteins and unique peptides characterized in a sample matrix by method without protein A chromatography and with protein A chromatography along with reproducibility statistics of the methods carried out according to exemplary embodiments.

FIG. 2 shows a protocol for the fractionation step carried out according to an exemplary embodiment.

FIG. 3 shows the number of proteins and unique peptides characterized in a sample matrix by method without a fractionation step and with a fractionation step along with reproducibility statistics of the methods carried out according to exemplary embodiments.

FIG. 4 shows the number of proteins and unique peptides characterized in a sample matrix by a method with a protein A chromatography step and a method with protein A chromatography step and a fractionation step along with reproducibility statistics of the methods carried out according to exemplary embodiments.

FIG. 5 shows the number of proteins and unique peptides characterized in a sample matrix by method wherein normal digestion of protein was carried out and a method wherein native digestion of protein was carried out along with reproducibility statistics of the methods carried out according to exemplary embodiments.

FIG. 6 shows the number of proteins and unique peptides characterized in a sample matrix subjected to native conditions by a method without protein A chromatography and a method with protein A chromatography along with reproducibility statistics of the methods carried out according to exemplary embodiments.

FIG. 7 shows the number of proteins and unique peptides characterized in a sample matrix by a method without FAIMS device and a method with FAIMS device along with reproducibility statistics of the methods carried out according to exemplary embodiments.

FIG. 8 shows the number of proteins and unique peptides characterized in a sample matrix by a method comprising protein A chromatography without FAIMS device and with FAIMS device along with reproducibility statistics of the methods carried out according to exemplary embodiments.

FIG. 9 shows the number and overlap of HCPs detected in an analysis according to an exemplary embodiment of (A) native vs. normal digests, (B) normal vs. protein A depleted digests, (C) native vs. protein A depleted native digests, (D) protein A depleted native digests with and without FAIMS, and (E) the optimized method vs. HCPs reported. All identified proteins have 2+unique peptides with a 1% peptide FDR and 5% protein FDR.

FIG. 10 shows a sample run with and without FAIMS conducted according to an exemplary embodiment: (A) the base peak chromatograms for a sample run with FAIMS (blue, red, and green) and without FAIMS (grey) with insert showing DS interference of HCP peptide, (B) fragmentation spectra of “revealed” HCP peptide. The peptides sequences include

(SEQ ID NO. 1)
K.KLEELDLDEQQR.K,
(SEQ ID NO. 2)
K.VYACEVTHQGLSSPVTK.S,
and
(SEQ ID NO. 3)
KLEELDLDEQQR

FIG. 11 shows the number and overlap of HCPs detected in replicate runs for all combinations of methods tried according to exemplary embodiments. All identified proteins have 2+unique peptides with a 1% peptide FDR and 5% protein FDR.

FIG. 12 shows number and overlap of HCPs detected in the protein A depleted native digest sample using FAIMS (A) compared to all other methods (B-H). All identified proteins have 2 or more unique peptides with a 1% peptide FDR and 5% protein FDR.

FIG. 13 shows a workflow of an exemplary embodiment.

### DETAILED DESCRIPTION

Host cell proteins (HCPs) are a class of impurities that must be removed from all cell-derived protein therapeutics. During cell-based production of these therapeutic proteins, the final protein based drug product must be highly purified so that impurities from cells are at acceptable low levels before clinical use. The impurities, in particular, host cell proteins (HCPs) derived from mammalian expression system (e.g., Chinese hamster ovary (CHO) cells) are required to be monitored. The general guidelines for total HCP levels in the final drug substance are less than 100 ppm (John H. Chon & Gregory Zarbis-Papastoitsis, Advances in the production and downstream processing of antibodies, 28 NEW BIOTECHNOLOGY 458-463 (2011)). HCPs are a concern for both patient safety and drug efficacy. See Leslie C. Eaton, Host cell contaminant protein assay development for recombinant biopharmaceuticals, 705 JOURNAL OF CHROMATOGRAPHY A 105-114 (1995); Xing Wang, Alan K. Hunter & Ned M. Mozier, Host cell proteins in biologics development: Identification, quantitation and risk assessment, 103 BIOTECHNOLOGY AND BIOENGINEERING 446-458 (2009); and Christina L. Zuch De Zafra et al., Host cell proteins in biotechnology-derived products: A risk assessment framework, 112 BIOTECHNOLOGY AND BIOENGINEERING 2284-2291 (2015). While HCP levels below 100 ppm are generally viewed as acceptable, the risk associated with a particular contaminant should be assessed individually and can necessitate an even lower limit of detection (Daniel G. Bracewell, Richard Francis & C. Mark Smales, The future of host cell protein (HCP) identification during process development and manufacturing linked to a risk-based management for their control, 112 BIOTECHNOLOGY AND BIOENGINEERING 1727-1737 (2015); Tanja Wolter & Andreas Richter, Assays for controlling host-cell impurities in biopharmaceuticals, 40 BIOPROCESS INTERNATIONAL 40-46 (2005).

Numerous reported cases describe the degradation of therapeutic proteins or stabilizing agents due to HCP activity (Nitin Dixit et al., Residual Host Cell Protein Promotes Polysorbate 20 Degradation in a Sulfatase Drug Product Leading to Free Fatty Acid Particles, 105 JOURNAL OF PHARMACEUTICAL SCIENCES1657-1666 (2016); Troii Hall et al., Polysorbates 20 and 80 Degradation by Group XV Lysosomal Phospholipase A2 Isomer X1 in Monoclonal Antibody Formulations., 105 JOURNAL OF PHARMACEUTICAL SCIENCES 1633-1642; Sharon X. Gao et al., Fragmentation of a highly purified monoclonal antibody attributed to residual CHO cell protease activity, 108 BIOTECHNOLOGY AND BIOENGINEERING 977-982 (2010); Deepti Ahluwalia et al., Identification of a host cell protein impurity in therapeutic protein, P1, 141 JOURNAL OF PHARMACEUTICAL AND BIOMEDICAL ANALYSIS 32-38 (2017); Amareth Lim et al., Characterization of a cathepsin D protease from CHO cell-free medium and mitigation of its impact on the stability of a recombinant therapeutic protein, 34 BIOTECHNOLOGY PROGRESS 120-129 (2017)).

The FDA does not specify a maximum acceptable level of HCP, but HCP concentrations in the final drug product must be controlled and reproducible from batch to batch (FDA, 1999). However, even when total HCP impurities are present at low levels in a drug substance, the trace amount of HCPs may not be acceptable for some particular HCPs that may cause an immune response, being toxic or biologically active after injection (J. R. Bierich, Treatment of Pituitary Dwarfism with Biosynthetic Growth Hormone, 75 ACTA PAEDIATRICA 13-18 (1986); T. Romer et al., Efficacy and safety of a new ready-to-use recombinant human growth hormone solution, 30 JOURNAL OF ENDOCRINOLOGICAL INVESTIGATION 578-589 (2007); Daniel G. Bracewell, Richard Francis & C. Mark Smales, The future of host cell protein (HCP) identification during process development and manufacturing linked to a risk-based management for their control, 112 BIOTECHNOLOGY AND BIOENGINEERING 1727-1737 (2015); Saloumeh Kadkhodayan Fischer et al., Specific Immune Response to Phospholipase B-Like 2 Protein, a Host Cell Impurity in Lebrikizumab Clinical Material, 19 THE AAPS JOURNAL 254-263 (2016); Andres H. Gutiérrez, Leonard Moise & Annie S. De Groot, Of [hamsters] and men, 8 HUMAN VACCINES & IMMUNOTHERAPEUTICS 1172-1174 (2012); Vibha Jawa et al., Evaluating Immunogenicity Risk Due to Host Cell Protein Impurities in Antibody-Based Biotherapeutics, 18 THE AAPS JOURNAL 1439-1452 (2016); Naghmeh Abiri et al., Assessment of the immunogenicity of residual host cell protein impurities of OsrHSA, 13 PLOS ONE (2018)). It may also be intolerable if HCPs pertain the potency to degrade antibody or alter the antibody binding potency (Nitin Dixit et al., Residual Host Cell Protein Promotes Polysorbate 20 Degradation in a Sulfatase Drug Product Leading to Free Fatty Acid Particles, 105 JOURNAL OF PHARMACEUTICAL SCIENCES1657-1666 (2016); Troii Hall et al., Polysorbates 20 and 80 Degradation by Group XV Lysosomal Phospholipase A2 Isomer X1 in Monoclonal Antibody Formulations., 105 JOURNAL OF PHARMACEUTICAL SCIENCES 1633-1642)). Therefore, it can be desirable to have methods that are able to monitor all HCP components individually.

Traditionally, the enzyme-linked immunosorbent assay (ELISA) with polyclonal anti-HCP antibodies has been used to quantify the overall HCPs abundance (Denise C. Krawitz et al., Proteomic studies support the use of multi product immunoassays to monitor host cell protein impurities, 6 PROTEOMICS 94-110 (2006); Catherine Em Hogwood, Daniel G Bracewell & C Mark Smales, Host cell protein dynamics in recombinant CHO cells, 4 BIOENGINEERED 288-291 (2013); Anne Luise Tscheliessnig et al., Host cell protein analysis in therapeutic protein bioprocessing—methods and applications, 8 BIOTECHNOLOGY JOURNAL 655-670 (2013)). Given the demand for measures of individual HCP components, ELISA might not be the final solution for evaluating level of HCPs. In addition, some weakly or non-immunogenic HCPs may not generate antibodies for ELISA detection, these HCPs are therefore not able to be detected. While ELISA is useful as an in-process control and release test, it has several important limitations including: measuring only total HCP levels, an inability to detect new sources of contamination, and a bias towards more immunogenic proteins (Fengqiang Wang, Daisy Richardson, & Mohammed Shameem, Host-cell protein measurement and control, 28 BIOPROCESS INTERNATIONAL 32-38 (2015); Judith Zhu-Shinioni et al., Host cell protein testing by ELISAs and the use of orthogonal methods, 111 BIOTECHNOLOGY AND BIOENGINEERING-2367-2379 (2014)). Another complication is that ELISA is typically reliant on antigens generated from cell lines lacking the therapeutic protein (null strains) which may have a substantially different HCP profile than the production strain. Additionally, HCPs that copurify with the therapeutic protein, of which there are many (See Kabila Aboulaich et al., A novel approach to monitor clearance of host cell proteins associated with monoclonal antibodies, 30 BIOTECHNOLOGY PROGRESS 1114-1124 (2014); Nicholas E. Levy et al., Identification and characterization of host cell protein product-associated impurities in monoclonal antibody bioprocessing, 111 BIOTECHNOLOGY AND BIOENGINEERING 904-912 (2013); Nicholas E. Levy et al., Host cell protein impurities in chromatographic polishing steps for monoclonal antibody purification, 113 BIOTECHNOLOGY AND BIOENGINEERING-1260-1272 (2015) may exhibit a non-linear response. If the HCP concentration in a sample is much higher than the null strain and there are insufficient antibodies capable of recognizing it, this can potentially lead to an underestimation of contaminants. Furthermore, not all HCPs can be detected by ELISA as not all proteins are immunogenic and consequently lack an associated antibody. Regulatory agencies are aware of these limitations and now expect orthogonal methods capable of detecting specific contaminants prior to widespread drug production. Indeed, complimentary HCP detection methods are now routinely employed not only for better oversight but for the substantial improvements to process development that such techniques can provide (Viktor Háda et al., Recent advancements, challenges, and practical considerations in the mass spectrometry-based analytics of protein biotherapeutics: A viewpoint from the biosimilar industry, 161 JOURNAL OF PHARMACEUTICAL AND BIOMEDICAL ANALYSIS 214-238 (2018); Kristin N Valente et al., Applications of proteomic methods for CHO host cell protein characterization in biopharmaceutical manufacturing, 53 CURRENT OPINION IN BIOTECHNOLOGY 144-150 (2018); and Matthew R. Schenauer, Gregory C. Flynn & Andrew M. Goetze, Identification and quantification of host cell protein impurities in biotherapeutics using mass spectrometry, 428 ANALYTICAL BIOCHEMISTRY 150-157 (2012)).

A number of complementary analytical approaches have been employed to monitor HCPs, including 1D/2D-PAGE and mass spectrometry based analytical technology (Julita K. Grzeskowiak et al., Two-dimensional fluorescence difference gel electrophoresis for comparison of affinity and non-affinity based downstream processing of recombinant monoclonal antibody, 1216 JOURNAL OF CHROMATOGRAPHY A 4902-4912 (2009); Catalin Doneanu et al., Analysis of host-cell proteins in biotherapeutic proteins by comprehensive online two-dimensional liquid chromatography/mass spectrometry, 4 MABS 24-44 (2012); Mi Jin et al., Profiling of host cell proteins by two-dimensional difference gel electrophoresis (2D-DIGE): Implications for downstream process development, 105 BIOTECHNOLOGY AND BIOENGINEERING3 06-316 (2010)). Liquid chromatography coupled with tandem mass spectrometry (LC-MS/MS) can also provide a means for both identification and quantification of HCP impurities simultaneously and has emerged as the major orthogonal method to complement the ELISA assay. However, a major challenge for mass spectrometry-based methods can be that the mass spectrometer by itself lacks the capability to detect the low concentration of HCPs when mixed with overwhelming and highly concentrated antibody drug substance. To overcome the issue of wide dynamic range (over 6 order of magnitude) between low ppm level HCPs and the high abundance therapeutic antibody, one strategy is to resolve the co-eluting peptides before mass spectrometry analysis, by adding another dimension of separation such as 2D-LC and ion mobility in addition to data-dependent acquisition or data-independent acquisition to increase the separation efficiency. In one study, Ecker et al. reported single digit ppm level HCP identification using LC-MS/MS with data independent acquisition and they also established a library including masses, retention times and fragment ions for the HCPs from null strains. Although this method is sensitive, it may lose the HCPs that are only co-expressed with certain product (Dawn M Ecker, Susan Dana Jones & Howard L Levine, The therapeutic monoclonal antibody market, 7 MABS 9-14 (2014)). Another study showed the capability of identifying 10 to 50 ppm HCPs using 2D-HPLC (Catalin Doneanu et al., Analysis of host-cell proteins in biotherapeutic proteins by comprehensive online two-dimensional liquid chromatography/mass spectrometry, 4 MABS 24-44 (2012); Donald E. Walker et al., A modular and adaptive mass spectrometry-based platform for support of bioprocess development toward optimal host cell protein clearance, 9 MABS 654-663 (2017)). However, the cycle times of 2D-LC are very long, and this method may not be not sensitive enough for lower levels of HCPs (<10 ppm) analysis. Additionally, this generally prevents the identification of novel contaminants, reducing its usefulness (although there are alternatives that may limit this shortcoming) (Veronika Reisinger et al., A mass spectrometry-based approach to host cell protein identification and its application in a comparability exercise, 463 ANALYTICAL BIOCHEMISTRY 1-6 (2014); Simion Kreimer et al., Host Cell Protein Profiling by Targeted and Untargeted Analysis of Data Independent Acquisition Mass Spectrometry Data with Parallel Reaction Monitoring Verification, 89 ANALYTICAL CHEMISTRY 5294-5302 (2017)).

Multidimensional chromatography has also been shown to improve sensitivity by providing better separation of HCP tryptic peptides from those of the therapeutic protein (See Catalin Doneanu et al., supra; Matthew R. Schenauer et al., supra; G. Joucla et al., Cation exchange versus multimodal cation exchange resins for antibody capture from CHO supernatants: Identification of contaminating Host Cell Proteins by mass spectrometry, 942-943 JOURNAL OF CHROMATOGRAPHY B 126-133 (2013; Qingchun Zhang et al., Comprehensive tracking of host cell proteins during monoclonal antibody purifications using mass spectrometry, 6 MABS 659-670 (2014); Amy Farrell et al., Quantitative Host Cell Protein Analysis Using Two Dimensional Data Independent LC-MSE, 87 ANALYTICAL CHEMISTRY 9186-9193 (2015); Feng Yang et al., A 2D LC-MS/MS Strategy for Reliable Detection of 10-ppm Level Residual Host Cell Proteins in Therapeutic Antibodies, 90 ANALYTICAL CHEMISTRY 13365-13372 (2018); Regina Kufer et al., Evaluation of Peptide Fractionation and Native Digestion as Two Novel Sample Preparation Workflows to Improve HCP Characterization by LC-MS/MS, 91 ANALYTICAL CHEMISTRY 9716-9723 (2019)). For example, high-pH offline fractionation can be combined with low-pH reversed-phase chromatography to greatly reduce sample complexity. However, both offline and online multidimensional chromatography cannot completely negate interference from therapeutic proteins and can significantly reduce sample throughput, making them unsuitable for routine analysis during production. Ion mobility, although rarely used for HCP analysis, can potentially provide additional separation without reducing sample throughput (See Catalin Doneanu et al., supra)

The other strategies focus on sample matrix preparation to enrich HCPs by removing the antibody in the sample matrix with affinity purification, limited digestion or by capturing HCPs using polyclonal antibodies (Lihua Huang et al., A Novel Sample matrix Preparation for Shotgun Proteomics Characterization of HCPs in Antibodies, 89 ANALYTICAL CHEMISTRY 5436-5444 (2017); Jenny Heidbrink Thompson et al., Improved detection of host cell proteins (HCPs) in a mammalian cell-derived antibody drug using liquid chromatography/mass spectrometry in conjunction with an HCP-enrichment strategy, 28 RAPID COMMUNICATIONS IN MASS SPECTROMETRY 855-860 (2014); James A Madsen et al., Toward the complete characterization of host cell proteins in biotherapeutics via affinity depletions, LC-MS/MS, and multivariate analysis, 7 MABS 1128-1137 (2015)). Removal of the therapeutic protein can improve detection of HCPs by several orders of magnitude but risks biasing results or unintentionally removing HCPs from the sample.

One of the major challenges for the existing methods can be a lack of capability to detect low concentrations of HCPs in a sample matrix (for example, 0.01-10 ppm) with a wide dynamic range (5-8 order) between HCP and drug which can cause the HCP signal to be suppressed in the analysis.

The ability to measure and monitor thousands of HCPs proportionally increases the amount of data acquired. Significant benefits exist if the information can be used to determine critical HCPs and thereby create an improved basis for risk management. The development of such a library of HCPs can be advantageous for in-house HCP screening, regulating and monitoring impurities in biopharmaceutical processes and to find newer targets for drug discovery. The HCP library can also be used to validate the identity of low abundance HCPs in the drug substance or throughout the purification process by comparing tandem mass spectra and protein identities with those confirmed to be present in the library. A DIA library for future analyses can be constructed from the masses, retention times, and fragment ions obtained from such a large number of HCPs.

Considering the limitations of existing methods, an effective and efficient method for identification of HCPs was developed.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described. All publications mentioned are hereby incorporated by reference.

The term “a” should be understood to mean “at least one”; and the terms “about” and “approximately” should be understood to permit standard variation as would be understood by those of ordinary skill in the art; and where ranges are provided, endpoints are included.

In some exemplary embodiments, the disclosure provides methods for characterizing a host-cell protein. As used herein, the term “host-cell protein” includes protein derived from the host cell and can be unrelated to the desired protein of interest. Host-cell protein can be a process-related impurity which can be derived from the manufacturing process and can include the three major categories: cell substrate-derived, cell culture-derived and downstream derived. Cell substrate-derived impurities include, but are not limited to, proteins derived from the host organism and nucleic acid (host cell genomic, vector, or total DNA). Cell culture-derived impurities include, but are not limited to, inducers, antibiotics, serum, and other media components. Downstream-derived impurities include, but are not limited to, enzymes, chemical and biochemical processing reagents (e.g., cyanogen bromide, guanidine, oxidizing and reducing agents), inorganic salts (e.g., heavy metals, arsenic, nonmetallic ion), solvents, carriers, ligands (e.g., monoclonal antibodies), and other leachables.

In some exemplary embodiments, the host-cell protein can have a pI in the range of about 4.5 to about 9.0. In one aspect, the pI can be about 4.5, about 5.0, about 5.5, about 5.6, about 5.7, about 5.8, about 5.9, about 6.0, about 6.1 about 6.2, about 6.3, about 6.4, about 6.5, about 6.6, about 6.7, about 6.8, about 6.9, about 7.0, about 7.1 about 7.2, about 7.3, about 7.4, about 7.5, about 7.6, about 7.7, about 7.8, about 7.9, about 8.0, about 8.1 about 8.2, about 8.3, about 8.4, about 8.5, about 8.6, about 8.7, about 8.8, about 8.9, or about 9.0.

In some exemplary embodiments, the disclosure provides methods for characterizing a host-cell protein in a sample matrix. In one aspect, the sample matrix can be obtained from any step of the bioprocess, such as, culture cell culture fluid (CCF), harvested cell culture fluid (HCCF), process performance qualification (PPQ), any step in the downstream processing, drug substance (DS), or a drug product (DP) comprising the final formulated product. In another aspect, the sample matrix can be selected from any step of the downstream process of clarification, chromatographic purification, viral inactivation, or filtration. In one other aspect, the drug product can be selected from manufactured drug product in the clinic, shipping, storage, or handling.

In some exemplary embodiments, the types of host-cell proteins in the composition can be at least two.

In some exemplary embodiments, the sample matrix can further comprise a protein of interest. As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. “Synthetic peptides or polypeptides” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may contain one or multiple polypeptides to form a single functioning biomolecule. A protein can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Another exemplary aspect, a protein can include antibody fragments, nanobodies, recombinant antibody chimeras, cytokines, chemokines, peptide hormones, and the like. Proteins may be produced using recombinant cell-based production systems, such as the insect bacculovirus system, yeast systems (e.g., Pichia sp.), mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation,” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS147-176 (2012)). In one aspect, proteins comprise modifications, adducts, and other covalently linked moieties. Those modifications, adducts and moieties include for example avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as, globular proteins and fibrous proteins; conjugated proteins, such as, nucleoproteins, glycoproteins, mucoproteins, chromoproteins, phosphoproteins, metalloproteins, and lipoproteins; and derived proteins, such as, primary derived proteins and secondary derived proteins. In one aspect, the protein of interest can be an antibody, a bispecific antibody, a multi-specific antibody, an antibody fragment, a monoclonal antibody, a fusion protein, or combinations thereof.

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, C_{H}1, C_{H}2 and C_{H}3. Each light chain comprises a light chain variable region (abbreviated herein as LCVR or VL) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, and FR4. In different embodiments of the invention, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment contains sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The phrase “bispecific antibody” includes an antibody capable of selectively binding two or more epitopes. Bispecific antibodies generally comprise two different heavy chains, with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain.

A typical bispecific antibody has two heavy chains each having three heavy chain CDRs, followed by a C_{H}1 domain, a hinge, a C_{H}2 domain, and a C_{H}3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats, such as, but not limited to triomab, knobs into holes IgG (kih IgG), crossMab, orth-Fab IgG, Dual-variable domains Ig (DVD-Ig), Two-in-one or dual action Fab (DAF), IgG-single-chain Fv (IgG-scFv), or κλ-bodies. The non-IgG-like different formats include Tandem scFvs, Diabody format, Single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES265-310 (2014)).

The methods of producing bsAbs are not limited to quadroma technology based on the somatic fusion of two different hybridoma cell lines, chemical conjugation, which involves chemical cross-linkers, and genetic approaches utilizing recombinant DNA technology. Examples of bsAbs include those disclosed in the following patent applications, which are hereby incorporated herein by reference: U.S. Ser. No. 12/823838, filed Jun. 25, 2010; U.S. Ser. No. 13/488628, filed Jun. 5, 2012; U.S. Ser. No. 14/031075, filed Sep. 19, 2013; U.S. Ser. No. 14/808171, filed Jul. 24, 2015; U.S. Ser. No. 15/713574, filed Sep. 22, 2017; U.S. Ser. No. 15/713569, field Sep. 22, 2017; U.S. Ser. No. 15/386453, filed Dec. 21, 2016; U.S. Ser. No. 15/386443, filed Dec. 21, 2016; U.S. Ser. No. 15/22343 filed Jul. 29, 2016; and U.S. Ser. No. 15/814,095, filed Nov. 15, 2017. Low levels of homodimer impurities can be present at several steps during the manufacturing of bispecific antibodies. The detection of such homodimer impurities can be challenging when performed using intact mass analysis due to low abundances of the homodimer impurities and the co-elution of these impurities with main species when carried out using a regular liquid chromatographic method.

As used herein “multi-specific antibody” or “Mab” refers to an antibody with binding specificities for at least two different antigens. While such molecules normally will only bind two antigens (i.e. bispecific antibodies, bsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

In some exemplary embodiments, the protein of interest can have a pI in the range of about 4.5 to about 9.0. In one aspect, the pI can be about 4.5, about 5.0, about 5.5, about 5.6, about 5.7, about 5.8, about 5.9, about 6.0, about 6.1 about 6.2, about 6.3, about 6.4, about 6.5, about 6.6, about 6.7, about 6.8, about 6.9, about 7.0, about 7.1 about 7.2, about 7.3, about 7.4, about 7.5, about 7.6, about 7.7, about 7.8, about 7.9, about 8.0, about 8.1 about 8.2, about 8.3, about 8.4, about 8.5, about 8.6, about 8.7, about 8.8, about 8.9, or about 9.0.

In some exemplary embodiments, the types of protein of interest in the sample matrix can be at least two. In one aspect, one of the at least two protein of interest can be a monoclonal antibody, a polyclonal antibody, a bispecific antibody, an antibody fragment, a fusion protein, or an antibody-drug complex. In some other embodiments, concentration of one of the at least two protein of interest can be about 20 mg/mL to about 400 mg/mL. In some exemplary embodiments, the types of protein of interest in the compositions are two. In some exemplary embodiments, the types of protein of interest in the compositions are three. In some exemplary embodiments, the types of protein of interest in the compositions are five.

In some exemplary embodiments, the two or more protein of interest in the composition can be selected from trap proteins, chimeric receptor Fc-fusion proteins, chimeric proteins, antibodies, monoclonal antibodies, polyclonal antibodies, human antibodies, bispecific antibodies, multi-specific antibodies, antibody fragments, nanobodies, recombinant antibody chimeras, cytokines, chemokines, or peptide hormones.

In some exemplary embodiments, the sample matrix can be a co-formulation.

In some exemplary embodiments, the protein of interest can be purified from mammalian cells. The mammalian cells can be of human origin or non-human origin can include primary epithelial cells (e.g., keratinocytes, cervical epithelial cells, bronchial epithelial cells, tracheal epithelial cells, kidney epithelial cells and retinal epithelial cells), established cell lines and their strains (e.g., 293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LSI80 cells, LS174T cells, NCI-H-548 cells, RPMI2650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-1 cells, Y-1 cells, LLC-PKi cells, PK(15) cells, GHi cells, GH3 cells, L2 cells, LLC-RC 256 cells, MHiCi cells, XC cells, MDOK cells, VSW cells, and TH-I, B1 cells, BSC-1 cells, RAf cells, RK-cells, PK-15 cells or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, Midi cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDMiC3 cells, KLN205 cells, McCoy cells, Mouse L cells, Strain 2071 (Mouse L) cells, L-M strain (Mouse L) cells, L-MTK′ (Mouse L) cells, NCTC clones 2472 and 2555, SCC-PSA1 cells, Swiss/3T3 cells, Indian muntjac cells, SIRC cells, Cn cells, and Jensen cells, Sp2/0, NS0, NS1 cells or derivatives thereof).

In some exemplary embodiments, the method for characterizing a host-cell protein can comprise enriching host-cell proteins in the sample matrix by contacting the sample matrix with a chromatography support.

As used herein, the term “chromatography” refers to a process in which a chemical mixture carried by a liquid or gas can be separated into components as a result of differential distribution of the chemical entities as they flow around or over a stationary liquid or solid phase. Non-limiting examples of chromatography include traditional reversed phase (RP), ion exchange (IEX) and normal phase chromatography (NP). Unlike RP, NP and IEX chromatography, in which hydrophobic interaction, hydrophilic interaction and ionic interaction respectively are the dominant interaction modes, mixed-mode chromatography can employ a combination of two or more of these interaction modes. Several types of liquid chromatography can be used with the mass spectrometer, such as, rapid resolution liquid chromatography (RRLC), ultra-performance liquid chromatography (UPLC), ultra-fast liquid chromatography (UFLC) and nano liquid chromatography (nLC). For further details on chromatography method and principles, see Colin et al. (Colin F. Poole et al., LIQUID CHROMATOGRAPHY FUNDAMENTALS AND INSTRUMENTATION (2017)).

In some exemplary embodiments, the chromatography support can be a liquid chromatography support. As used herein, the term “liquid chromatography” refers to a process in which a chemical mixture carried by a liquid can be separated into components as a result of differential distribution of the chemical entities as they flow around or over a stationary liquid or solid phase. Non-limiting examples of liquid chromatography include reversed phase liquid chromatography, ion-exchange chromatography, size exclusion chromatography, affinity chromatography, mixed-mode chromatography or hydrophobic chromatography.

As used herein, “ion exchange chromatography” can include separations including any method by which two substances are separated based on the difference in their respective ionic charges, either on the molecule of interest and/or chromatographic material as a whole or locally on specific regions of the molecule of interest and/or chromatographic material, and thus can employ either cationic exchange material or anionic exchange material. Ion exchange chromatography separates molecules based on differences between the local charges of the molecules of interest and the local charges of the chromatographic material. A packed ion-exchange chromatography column or an ion-exchange membrane device can be operated in a bind-elute mode, a flow-through, or a hybrid mode. After washing the column or the membrane device with the equilibration buffer or another buffer with different pH and/or conductivity, the product recovery can be achieved by increasing the ionic strength (e.g., conductivity) of the elution buffer to compete with the solute for the charged sites of the ion exchange matrix. Changing the pH and thereby altering the charge of the solute can be another way to achieve elution of the solute. The change in conductivity or pH may be gradual (gradient elution) or stepwise (step elution). The column can be then regenerated before next use. Anionic or cationic substituents may be attached to matrices in order to form anionic or cationic supports for chromatography. Non-limiting examples of anionic exchange substituents include diethylaminoethyl (DEAE), quaternary aminoethyl (QAE) and quaternary amine (Q) groups. Cationic substituents include carboxymethyl (CM), sulfoethyl (SE), sulfopropyl (SP), phosphate (P) and sulfonate (S). Cellulose ion exchange medias or support can include DE23™, DE32™, DE52™, CM-23™, CM-32™, and CM-52™ are available from Whatman Ltd. Maidstone, Kent, U.K. SEPHADEX®-based and -locross-linked ion exchangers are also known. For example, DEAE-, QAE-, CM-, and SP-SEPHADEX® and DEAE-, Q-, CM- and S-SEPHAROSE® and SEPHAROSE® Fast Flow, and Capto™ S are all available from GE Healthcare. Further, both DEAE and CM derivatized ethylene glycol-methacrylate copolymer such as TOYOPEARL™ DEAE-650S or M and TOYOPEARL™ CM-650S or M are available from Toso Haas Co., Philadelphia, Pa., or Nuvia S and UNOSphere™ S from BioRad, Hercules, Calif, Eshmuno® S from EMD Millipore, Mass.

As used herein, the term “hydrophobic interaction chromatography resin” can include a solid phase which can be covalently modified with phenyl, octyl, or butyl chemicals. It can use the properties of hydrophobicity to separate molecules from one another. In this type of chromatography, hydrophobic groups such as, phenyl, octyl, hexyl or butyl can be attached to the stationary column. Molecules that pass through the column that have hydrophobic amino acid side chains on their surfaces are able to interact with and bind to the hydrophobic groups on the column. Examples of hydrophobic interaction chromatography resins or support include Phenyl sepharose FF, Capto Phenyl (GE Healthcare, Uppsala, Sweden), Phenyl 650-M (Tosoh Bioscience, Tokyo, Japan) and Sartobind Phenyl (Sartorius corporation, New York, USA).

As used herein, the term “Mixed Mode Chromatography (MMC)” or “multimodal chromatography” includes a chromatographic method in which solutes interact with stationary phase through more than one interaction mode or mechanism. M1VIC can be used as an alternative or complementary tool to traditional reversed phase (RP), ion exchange (IEX) and normal phase chromatography (NP). Unlike RP, NP and IEX chromatography, in which hydrophobic interaction, hydrophilic interaction and ionic interaction respectively are the dominant interaction modes, mixed-mode chromatography can employ a combination of two or more of these interaction modes. Mixed mode chromatography media can provide unique selectivity that cannot be reproduced by single mode chromatography. Mixed mode chromatography can also provide potential cost savings, longer column lifetimes and operation flexibility compared to affinity-based methods. In some exemplary embodiments, the mixed mode chromatography media can be comprised of mixed mode ligands coupled to an organic or inorganic support, sometimes denoted a base matrix, directly or via a spacer. The support may be in the form of particles, such as essentially spherical particles, a monolith, filter, membrane, surface, capillaries, etc. In some specific exemplary embodiments, the support can be prepared from a native polymer, such as cross-linked carbohydrate material, such as agarose, agar, cellulose, dextran, chitosan, konjac, carrageenan, gellan, alginate etc. To obtain high adsorption capacities, the support can be porous, and ligands are then coupled to the external surfaces as well as to the pore surfaces. Such native polymer supports can be prepared according to standard methods, such as inverse suspension gelation (Stellan Hjertén, The preparation of agarose spheres for chromatography of molecules and particles, 79 BIOCHIMICA ET BIOPHYSICA ACTA (BBA)—BIOPHYSICS INCLUDING PHOTOSYNTHESIS 393-398 (1964) incorporated herein by reference). Alternatively, the support can be prepared from a synthetic polymer, such as cross-linked synthetic polymers, e.g., styrene or styrene derivatives, divinylbenzene, acrylamides, acrylate esters, methacrylate esters, vinyl esters, vinyl amides etc. Such synthetic polymers can be produced according to standard methods, see e.g., Eduardo Vivaldo-Lima et al., An Updated Review on Suspension Polymerization, 36 INDUSTRIAL & ENGINEERING CHEMISTRY RESEARCH 939-965 (1997). Porous native or synthetic polymer supports are also available from commercial sources, such as Amersham Biosciences, Uppsala, Sweden.

In some exemplary embodiments, the method for characterizing a host-cell protein can comprise enriching host-cell proteins in the sample matrix by contacting the sample matrix with an affinity chromatography support.

As used herein, “affinity chromatography” can include separations including any method by which two substances are separated based on their affinity to a chromatographic material. Non-limiting examples of affinity chromatography support include, but are not limited to Protein A resin, Protein G resin, affinity supports comprising the antigen against which the binding molecule was raised, and affinity supports comprising an Fc binding protein. The affinity chromatography resin can be formed by immobilizing Protein A, Protein G, antigen against which the binding molecule was raised, or Fc binding protein on a resin, such as, agarose or sepharose. There are several commercial sources for Protein A resin. Non-limiting examples of Protein A resin include Mab Select SuRe™, Mab Select SuRe LX, MabSelect, Mab Select Xtra, rProtein A Sepharose from GE Healthcare, and ProSep HC, ProSep Ultra, and ProSep Ultra Plus from EMD Millipore.

In one aspect, the affinity chromatographic material can be equilibrated with a suitable buffer prior to sample matrix loading. Following this equilibration, the sample matrix can be loaded onto the column. In one aspect, following the loading of the affinity chromatographic material, the affinity chromatographic material can be washed one or multiple times using an appropriate wash buffer. In some specific aspects, a flow-through from the wash can be collected. In some specific aspects, the flow-through from the wash can be further processed. Optionally other washes, including washes employing different buffers, can be employed prior to eluting the column. A flow-through from the washes can be collected and further processed. The affinity chromatographic material can also be eluted using an appropriate elution buffer. The eluate can be monitored using techniques well known to those skilled in the art. For example, the absorbance at OD280 can be followed. The elution fraction(s) of interest can then be prepared for further processing.

In one aspect, a kosmotropic salt solution can be supplemented into the sample matrix comprising the protein of interest prior to contacting with an affinity chromatography resin. The kosmotropic salt solution comprises at least one kosmotropic salt. Examples of suitable kosmotropic salts include, but are not limited to ammonium sulfate, sodium sulfate, sodium citrate, potassium sulfate, potassium phosphate, sodium phosphate and a combination thereof. In one aspect, the kosmotropic salt is ammonium sulfate; in another aspect, the kosmotropic salt is sodium sulfate; and in another aspect, the kosmotropic salt is sodium citrate. The kosmotropic salt is present in the kosmotropic salt solution at a concentration of from about 0.3 M to about 1.1 M. In one embodiment, the kosmotropic salt is present in the kosmotropic salt solution at a concentration of about 0.5 M.

In some exemplary embodiments, the enrichment step can further comprise treating a sample obtained from the chromatography support.

In some exemplary embodiments, the treatment can include adding a hydrolyzing agent to the sample to produce peptides. As used herein, the term “hydrolyzing agent” refers to any one or combination of a large number of different agents that can perform digestion of a protein. Non-limiting examples of hydrolyzing agents that can carry out enzymatic digestion include trypsin, endoproteinase Arg-C, endoproteinase Asp-N, endoproteinase Glu-C, outer membrane protease T (OmpT), immunoglobulin-degrading enzyme of Streptococcus pyogenes (IdeS), chymotrypsin, pepsin, thermolysin, papain, pronase, and protease from Aspergillus Saitoi. Non-limiting examples of hydrolyzing agents that can carry out non-enzymatic digestion include the use of high temperature, microwave, ultrasound, high pressure, infrared, solvents (non-limiting examples are ethanol and acetonitrile), immobilized enzyme digestion (IMER), magnetic particle immobilized enzymes, and on-chip immobilized enzymes. For a recent review discussing the available techniques for protein digestion see Switazar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, 12 JOURNAL OF PROTEOME RESEARCH 1067-1077 (2013)). One or a combination of hydrolyzing agents can cleave peptide bonds in a protein or polypeptide, in a sequence-specific manner, generating a predictable collection of shorter peptides.

The term ratio of hydrolyzing agent to the protein and the time required for digestion can be appropriately selected to obtain a digestion of the protein. When the enzyme to substrate ratio is unsuitably high, it can cause a non-specific cleavage (potentially breaking all proteins/peptides into individual amino acids) thereby limiting the ability to identify proteins as well as reducing sequence coverage. On the other hand, a low E/S ratio would need long digestion and thus long sample preparation time. The enzyme to substrate ratio can range from about 1:0.5 to about 1:500.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion.

One of the widely accepted methods for digestion of proteins in a sample involves the use of proteases. Many proteases are available and each of them have their own characteristics in terms of specificity, efficiency, and optimum digestion conditions. Proteases refer to both endopeptidases and exopeptidases, as classified based on the ability of the protease to cleave at non-terminal or terminal amino acids within a peptide. Alternatively, proteases also refer to the six distinct classes—aspartic, glutamic, and metalloproteases, cysteine, serine, and threonine proteases, as classified on the mechanism of catalysis. The terms “protease” and “peptidase” are used interchangeably to refer to enzymes which hydrolyze peptide bonds.

Apart from contacting a host-cell protein to a hydrolyzing agent, the method can optionally include steps for reducing the host-cell protein, alkylating the host-cell protein, buffering the host-cell protein, and/or desalting the sample matrix. These steps can be accomplished in any suitable manner as desired.

In some exemplary embodiments, the treatment can include adding a protein reducing agent to the sample. As used herein, the term “protein reducing agent” refers to the agent used for reduction of disulfide bridges in a protein. Non-limiting examples of the protein reducing agents used to reduce the protein are dithiothreitol (DTT), β-mercaptoethanol, Ellman's reagent, hydroxylamine hydrochloride, sodium cyanoborohydride, tris(2-carboxyethyl)phosphine hydrochloride (TCEP-HCl), or combinations thereof.

In some exemplary embodiments, the treatment can include adding a protein alkylating agent to the sample. As used herein, the term “protein alkylating agent” refers to the agent used for alkylate certain free amino acid residues in a protein. Non-limiting examples of the protein alkylating agents are iodoacetamide (IOA), chloroacetamide (CAA), acrylamide (AA), N-ethylmaleimide (NEM), methyl methanethiosulfonate (MMTS), and 4-vinylpyridine or combinations thereof.

In some exemplary embodiments, the treatment can include adding one or more form the group consisting of alkylating agent, reducing agent, hydrolyzing agent or combinations thereof. The additions of these agents to the sample can vary. The addition can be carried by adding the sample to the agents or by adding the agents to the samples.

In some exemplary embodiments, the method for characterizing a host-cell protein can comprise enriching host-cell proteins in the sample matrix by contacting the sample matrix with a chromatography support and performing a fractionation step. As used herein, the term “fractionation” can include a process of separating various peptides obtained from digesting the host-cell proteins present in a sample matrix. The process can involve separating the peptides using an appropriate peptide fractionation technique(s) which can fractionate the peptides based their various general properties such as the peptides' pI, hydrophobicity, metal binding ability, content of exposed thiol groups, size, charge, shape, solubility, stability and sedimentation velocity, ability to bind with various ionic groups, and affinity for substrates as a basis for isolating peptide(s) from complex biological sample matrixes. Peptides can also be separated based on their cellular location, thereby allowing to extract cytoplasmic, nuclear and membrane proteins.

In some exemplary embodiments, the fractionation can be a size-based fractionation. In one aspect, the size-based fractionation can be carried out by using gel electrophoresis. Details on gel electrophoresis can be found in Zaifang Zhu, Joann Lu & Shaorong Liu, Protein separation by capillary gel electrophoresis: A review, 709 ANALYTICA CHIMICA ACTA 21-31 (2012), which is incorporated herein by reference. Further principles and basics can be found in SAMEH MAGDELDIN, GEL ELECTROPHORESIS: PRINCIPLES AND BASICS (2012), which is incorporated herein by reference.

In one aspect, the size-based fractionation can be carried out by using dialysis. The dialysis can be performed using a molecular cut-off membrane filter or a series of membrane filters. The dialysis can also be performed using dialysis cassettes. Example of one such dialysis methods can include using Slide-A-Lyzer™ Dialysis Cassettes. The cassette design helps maximize surface area to sample volume ratio and enables excellent sample recoveries.

In one aspect, the size-based fractionation can be carried out by using capillary electrophoresis. Recent trends and advances on capillary electrophoresis can be found in Robert Voeten et al., Capillary Electrophoresis: Trends and Recent Advances, 90 ANALYTICAL CHEMISTRY 1464-1481 (2018) and Maria Ramos-Payan et al., Recent trends in capillary electrophoresis for complex samples analysis: A review, 39 ELECTROPHORESIS 111-125 (2017), which are incorporated herein by reference. Further principles and basics can be found in Harry Whatley, Basic Principles and Modes of Capillary Electrophoresis, CLINICAL AND FORENSIC APPLICATIONS OF CAPILLARY ELECTROPHORESIS 21-58, which is incorporated herein by reference.

In one aspect, the size-based fractionation can be carried out using size exclusion chromatography. The phrase “size exclusion chromatography” or “SEC” or “gel filtration” includes a liquid column chromatographic technique that can sort molecules according to their size in solution. As used herein, the terms “SEC chromatography resin” or “SEC chromatography media” are used interchangeably herein and can include any kind of solid phase used in SEC which separates the impurity from the desired product (e.g., a homodimer contaminant for a bispecific antibody product). The volume of the resin, the length and diameter of the column to be used, as well as the dynamic capacity and flow-rate can depend on several parameters such as the volume of fluid to be treated, concentration of protein in the fluid to be subjected to the process of the invention, etc. Determination of these parameters for each step is well within the average skills of the person skilled in the art. A brief practical review on size exclusion chromatography can be found in Richard R. Burgess, A brief practical review of size exclusion chromatography: Rules of thumb, limitations, and troubleshooting, 150 PROTEIN EXPRESSION AND PURIFICATION 81-85 (2018) and Gloria Brusotti et al., Advances on Size Exclusion Chromatography and Applications on the Analysis of Protein Biopharmaceuticals and Protein Aggregates: A Mini Review, 81 CHROMATOGRAPHIA 3-23 (2017), which are each incorporated herein by reference. Further principles and basics of SEC can be found in Paula Hong, Stephan Koza & Edouard S. P. Bouvier, A Review Size-Exclusion Chromatography For The Analysis Of Protein Biotherapeutics And Their Aggregates, 35 JOURNAL OF LIQUID CHROMATOGRAPHY & RELATED TECHNOLOGIES 2923-2950 (2012), which is incorporated herein by reference. Newer methods for size exclusion chromatography can also be used for the methods, as illustrated in Singh et al., New Automated Systems for Size fractionation of Protein Samples, 24 JOURNAL OF BIOMOLECULAR TECHNOLOGIES S60S61 (2013), which is incorporated herein by reference.

In one aspect, the size-based fractionation can be carried out using field flow fractionation. The field flow fractionation (FFF) is a class of ‘soft impact’ elution techniques employed mainly to separate heterogeneous mixtures of supramolecules, proteins and bioparticles (<100 μm dia.) within laminar microfluidic flows. An overview of the FFF is provided by in the article by Messaud et al. (Fathi A. Messaud et al., An overview on field-flow fractionation techniques and their applications in the separation and characterization of polymers, 34 PROGRESS IN POLYMER SCIENCE 351-368 (2009)), which is incorporated herein by reference. Further techniques for FFF can be found in T. Kowalkowski et al., Field-Flow Fractionation: Theory, Techniques, Applications and the Challenges, 36 CRITICAL REVIEWS IN ANALYTICAL CHEMISTRY 129-135 (2006) and Barbara Roda et al., Field-flow fractionation in bioanalysis: A review of recent trends, 635 ANALYTICA CHIMICA ACTA 132-143 (2009), which are each incorporated herein by reference.

In some exemplary embodiments, the fractionation can be a hydrophobicity-based fractionation. In one aspect, the size-based fractionation can be carried out using reversed phase chromatography. Reversed phase chromatography is the most widely used chromatographic mode allowing separation of proteins on the basis of their hydrophobicity. The separation is based on the analytes partition coefficient between the polar mobile phase and the hydrophobic (nonpolar) stationary phase. In the case of peptides, more polar peptides elute first while less polar peptides interact more strongly with the hydrophobic groups that form a liquid-like' layer around the solid silica support. RPLC has been extensively applied in peptide separation for its ease of use with gradient elution, compatibility with aqueous samples and versatility of the retention mechanism, allowing changes in the separation brought by changes in the pH, organic modifier or additives. In one aspect, the size-based fractionation can be carried out using a pH gradient chromatography.

In some exemplary embodiments, the reversed phase chromatography can comprise a low pH reversed phase liquid chromatography separation using the nano LC. In one aspect, the reversed phase chromatography can comprise a high pH reversed phase liquid chromatography separation. In a specific aspect, the reversed phase chromatography can comprise a high pH reversed phase liquid chromatography separation orthogonal to a low pH reversed phase liquid chromatography. An overview of one such two-Dimensional Separation Using High-pH and Low-pH Reversed Phase Liquid Chromatography for Top-down Proteomics can be found in Zhe Wang et al., Two-dimensional separation using high-pH and low-pH reversed phase liquid chromatography for top-down proteomics, 427 INTERNATIONAL JOURNAL OF MASS SPECTROMETRY 43-51 (2018).

In some exemplary embodiments, the fractionation can be a charge-based fractionation. In another aspect, the charge-based fractionation can be carried out using an ion-exchange chromatography. In a specific aspect, the ion-exchange chromatography can be a cation-exchange chromatography. In another specific aspect, the ion-exchange chromatography can be an anion-exchange chromatography.

In some exemplary embodiments, the fractionation can be a pI-based fractionation. In one aspect, the charge-based fractionation can be carried out using an ion-exchange chromatography. In a specific aspect, the ion-exchange chromatography can be a cation-exchange chromatography. In another specific aspect, the ion-exchange chromatography can be an anion-exchange chromatography. In one aspect, the charge-based fractionation can be carried by isoelectric focusing. Isoelectric focusing (IEF) can provide separation of proteins, wherein proteins can travel according to their charge under the influence of an electric field, in the presence of a pH gradient, until the net charge of the molecule is zero (e.g., isoelectric point, pI). The separation can be deemed according to the composition of amino acids and exposed charged residues, which behave as weak acids and bases. The migration of the proteins will follow basic principles of electrophoresis; however, the mobility will change in the presence of the pH gradient by slowing down migration at values close to the pI value. An overview of the IEF is provided by in the article by Pergande and Cologna Melissa Pergande & Stephanie Cologna, Isoelectric Point Separations of Peptides and Proteins, 5 PROTEOMES 4 (2017), which is incorporated herein by reference. Further techniques for IEF can be found in Findley Cornell, Isoelectric Focusing, Blotting and Probing Methods for Detection and Identification of Monoclonal Proteins, 30 THE CLINICAL BIOCHEMIST REVIEWS 123-130 (2009); Tomasz Bączek, Fractionation of peptides in proteomics with the use of pI-based approach and ZipTip pipette tips, 34 JOURNAL OF PHARMACEUTICAL AND BIOMEDICAL ANALYSIS 851-860 (2004); C. F. Ivory, A Brief Review of Alternative Electrofocusing Techniques, 35 Separation Science and Technology 1777-1793 (2000) ; G. B. Smejkal, Solution phase isoelectric fractionation in the multi-compartment electrolyser: A divide and conquer strategy for the analysis of complex proteomes, 4 BRIEFINGS IN FUNCTIONAL GENOMICS AND PROTEOMICS 76-81 (2005), and David Garfin, Gel Electrophoresis of Proteins, in ESSENTIAL CELL BIOLOGY, VOLUME 1, A PRACTICAL APPROACH 197-268 (2003), which are each incorporated herein by reference.

Further improvements in the pI-based fractionation can be used for the fractionation step, such as, methods delineated in Subhashini Selvaraju & Ziad El Rassi, Liquid-phase-based separation systems for depletion, prefractionation and enrichment of proteins in biological fluids and matrices for in-depth proteomics analysis—An update covering the period 2008-2011, 33 ELECTROPHORESIS 74-88 (2011).

In some exemplary embodiments, the method for characterizing a host-cell protein can comprise further characterizing at least one of the host-cell proteins using a mass spectrometer.

In some exemplary embodiments, the characterizing can include identifying the peptides obtained from the fractionation step. Peptide identification can be further performed by comparing the mass spectra derived from the polypeptide fragmentation with the theoretical mass spectra generated from in silico digestion of a protein. Protein inference is then accomplished by assigning peptide sequences to proteins.

As used herein, the term “mass spectrometer” includes a device capable of identifying specific molecular species and measuring their accurate masses. The term is meant to include any molecular detector into which a polypeptide or peptide may be eluted for detection and/or characterization. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends heavily on the application.

In some exemplary embodiments, the mass spectrometer can be a tandem mass spectrometer.

As used herein, the term “tandem mass spectrometry” includes a technique where structural information on sample matrix molecules is obtained by using multiple stages of mass selection and mass separation. A prerequisite is that the sample matrix molecules can be transferred into the gas phase and ionized intact and that they can be induced to fall apart in some predictable and controllable fashion after the first mass selection step. Multistage MS/MS, or MSⁿ, can be performed by first selecting and isolating a precursor ion (MS²), fragmenting it, isolating a primary fragment ion (MS³), fragmenting it, isolating a secondary fragment (MS⁴), and so on as long as one can obtain meaningful information or the fragment ion signal is detectable. Tandem MS has been successfully performed with a wide variety of analyzer combinations. What analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device.

The peptides identified by the mass spectrometer can be used as surrogate representatives of the intact protein and their post translational modifications. They can be used for protein characterization by correlating experimental and theoretical MS/MS data, the latter generated from possible peptides in a protein sequence database. The characterization includes, but is not limited to, sequencing amino acids of the protein fragments, determining protein sequencing, determining protein de novo sequencing, locating post-translational modifications, or identifying post translational modifications, or comparability analysis, or combinations thereof.

As used herein, the term “database” refers to bioinformatic tools which provide the possibility of searching the uninterpreted MS-MS spectra against all possible sequences in the database(s). Non-limiting examples of such tools are Mascot (http://www.matrixscience.com), Spectrum Mill (http://www.chem.agilent.com), PLGS (http://www.waters.com), PEAKS (http://www.bioinformaticssolutions.com), Proteinpilot (http://download.appliedbiosystems.com//proteinpilot), Phenyx (http://www.phenyx-ms.com), Sorcerer (http://www.sagenresearch.com), OMSSA (http://www.pubchem.ncbi.nlm.nih.gov/omssa/), X!Tandem (http://www.thegpm.org/TANDEM/), Protein Prospector (http://www. http://prospector.ucsf.edu/prospector/mshome.htm), Byonic (https://www.proteinmetrics.com/products/byonic), Andromeda (https://www.ncbi.nlm.nih.gov/pubmed/21254760) or Sequest (http://fields.scripps.edu/sequest).

In some exemplary embodiments, the mass spectrometer can be coupled to a liquid chromatography system. In another exemplary embodiments, the mass spectrometer can be coupled to a nano liquid chromatography. In one aspect, the mobile phase used to elute the protein in liquid chromatography can be a mobile phase that can be compatible with a mass spectrometer. In a specific aspect, the mobile phase can be ammonium acetate, ammonium bicarbonate, or ammonium formate, acetonitrile, water, formic acid, a volatile acid, or combinations thereof.

In some exemplary embodiments, the method for characterizing a host-cell protein can comprise further characterizing at least one of the host-cell proteins using High-Field Asymmetric Waveform Ion Mobility Spectrometry. As used herein, “High field asymmetric waveform ion mobility spectrometry” or “FAIMS” or “differential mobility spectrometry” or “DMS” can include an atmospheric pressure ion mobility technique that separates gas-phase ions by their behavior in strong and weak electric fields. FAIMS device can be easily interfaced with electrospray ionization and has been implemented as an additional separation mode between liquid chromatography (LC) and mass spectrometry (MS) in proteomic studies. FAIMS separation can be orthogonal to both LC and MS and can be used as a means of on-line fractionation to improve detection of peptides in complex samples. FAIMS can improve dynamic range and concomitantly the detection limits of ions by filtering out chemical noise. FAIMS can also be used to remove interfering ion species and to select peptide charge states optimal for identification by tandem MS. A review on use of FAIMS for mass spectrometry-based proteomics can be found in the article published by Swearingen and Moritz (Kristian E Swearingen & Robert L Moritz, High-field asymmetric waveform ion mobility spectrometry for mass spectrometry-based proteomics, 9 Expert Review of Proteomics 505-517 (2012)), which is incorporated herein by reference. Further details on FAIMS can also be found in several reviews: Roger Guevremont, High-field asymmetric waveform ion mobility spectrometry: A new tool for mass spectrometry, 1058 JOURNAL OF CHROMATOGRAPHY A 3-19 (2004); Alexandre A. Shvartsburg et al., Field Asymmetric Waveform Ion Mobility Spectrometry Studies of Proteins: Dipole Alignment in Ion Mobility Spectrometry?, 110 THE JOURNAL OF PHYSICAL CHEMISTRY B 21966-21980 (2006); Beata M. Kolakowski & Zoltán Mester, Review of applications of high-field asymmetric waveform ion mobility spectrometry (FAIMS) and differential mobility spectrometry (DMS), 132 THE ANALYST 842 (2007), all of which are incorporated herein by reference. A general review of FAIMS by Kolakowski and Mester, a series of theoretical and practical explorations of FAIMS by Nazarov and co-workers (Nazarov, Electric field dependence of the ion mobility, 285 INTERNATIONAL JOURNAL OF MASS SPECTROMETRY 149-156 (2009)); Bradley B. Schneider et al., Planar differential mobility spectrometer as a pre-filter for atmospheric pressure ionization mass spectrometry, 298 INTERNATIONAL JOURNAL OF MASS SPECTROMETRY 45-54 (2010); Evgeny V. Krylov et al., Selection and generation of waveforms for differential mobility spectrometry, 81 REVIEW OF SCIENTIFIC INSTRUMENTS 024101 (2010); Bradley B. Schneider et al., Control of Chemical Effects in the Separation Process of a Differential Mobility Mass Spectrometer System, 16 EUROPEAN JOURNAL OF MASS SPECTROMETRY 57-71 (2010); Stephen L. Coy et al., Detection of radiation-exposure biomarkers by differential mobility prefiltered mass spectrometry (DMS-MS), 291 INTERNATIONAL JOURNAL OF MASS SPECTROMETRY 108-117 (2010); Bradley B. Schneider et al., Control of Chemical Effects in the Separation Process of a Differential Mobility Mass Spectrometer System, 16 EUROPEAN JOURNAL OF MASS SPECTROMETRY 57-71 (2010) and a book by Shvartsburg (ALEXANDRE A. SHVARTSBURG, DIFFERENTIAL ION MOBILITY SPECTROMETRY NONLINEAR ION TRANSPORT AND FUNDAMENTALS OF FAIMS (2009)), all of which are incorporated herein by reference.

Any of the commercial or adapted mass spectrometers and FAIMS cells/systems/devices can be utilized for the characterization of the host-cell protein. The FAIMS cells can vary in size—can be a “full-size” cell (FS-FAIMS) with a length of 65 mm, width of 20 mm, and analytical gap of 2mm; and a “quarter-size” cell (QS-FAIMS) with a length of 15 mm, a width of 5 mm, and an analytical gap of 0.38 mm. The FAIMS device used can be c-FAIMS by Ionalytics or p-FAIMS by Sionex. Miniaturized, chip-based FAIMS systems can also be used, such as, obtained from Owlstone Nanotech Inc.: UltraFAIMS A1 and the Lonestar Gas Analyzer. Both chips in each device are comprised of two interdigitated electrodes that create a serpentine geometry across the face of the chip, where each row is a distinct planar FAIMS channel. The FAIMS Pro™ Interface from Thermo Scientific can also be used for the method.

In some exemplary embodiments, the method for characterizing host-cell proteins in a sample can comprise steps for enriching host-cell proteins in the sample by contacting the sample with an affinity chromatography support and performing a fractionation step.

In some exemplary embodiments, the method for characterizing host-cell proteins in a sample can comprise steps for enriching host-cell proteins in the sample by contacting the sample with a protein A chromatography support and performing a fractionation step.

In some exemplary embodiments, the method for characterizing host-cell proteins in a sample can comprise steps for enriching host-cell proteins in the sample by contacting the sample with a chromatography support, performing a fractionation step and characterizing at least one of the host-cell proteins using a mass spectrometer.

In some exemplary embodiments, the method for characterizing host-cell proteins in a sample can comprise steps for enriching host-cell proteins in the sample by contacting the sample with a chromatography support, performing a fractionation step and characterizing at least one of the host-cell proteins using a tandem mass spectrometer. In one aspect, the tandem mass spectrometer can be tandem-in-space or tandem-in-time.

In some exemplary embodiments, the method for characterizing host-cell proteins in a sample can comprise steps for enriching host-cell proteins in the sample by contacting the sample with a chromatography support, performing a fractionation step and characterizing at least one of the host-cell proteins using a mass spectrometer coupled to a liquid chromatography system. In one aspect, the liquid chromatography system can be a nano-liquid chromatography system (nLC).

In some exemplary embodiments, the method for characterizing host-cell proteins in a sample can comprise steps for enriching host-cell proteins in the sample by contacting the sample with a chromatography support, performing a fractionation step and characterizing at least one of the host-cell proteins using FAIMS-MS. In one aspect, the carrier gas used for FAIMS can include volatile chemical modifiers. In a specific aspect, the volatile chemical modifier can be isopropanol or methylene chloride.

In one specific exemplary embodiment, FAIMS device can be used on conjunction with MS. In another specific exemplary embodiment, FAIMS device can be used on conjunction with LC and MS.

In some exemplary embodiments, the method for characterizing host-cell proteins in a sample can comprise steps for enriching host-cell proteins in the sample by contacting the sample with a chromatography support, performing a fractionation step and characterizing at least one of the host-cell proteins using a LC-FAIMS-MS.

In some exemplary embodiments, the method for characterizing host-cell proteins in a sample can comprise steps for enriching host-cell proteins in the sample by contacting the sample with a chromatography support and performing a fractionation step, wherein the method can be capable of characterizing at least about 20% more host-cell proteins than a method comprising the enriching step and not the fractionation step. In one aspect, the method can be capable of characterizing at least about 20% more host-cell proteins, at least about 25% more host-cell proteins, at least about 30% more host-cell proteins, at least about 35% more host-cell proteins, at least about 40% more host-cell proteins, at least about 45% more host-cell proteins, at least about 50% more host-cell proteins, at least about 55% more host-cell proteins, at least about 60% more host-cell proteins, at least about 65% more host-cell proteins, at least about 70% more host-cell proteins, at least about 75% more host-cell proteins, at least about 80% more host-cell proteins, at least about 85% more host-cell proteins, at least about 90% more host-cell proteins, at least about 95% more host-cell proteins, at least about 100% more host-cell proteins, at least about 105% more host-cell proteins, at least about 110% more host-cell proteins, at least about 115% more host-cell proteins, at least about 120% more host-cell proteins, at least about 125% more host-cell proteins, at least about 130% more host-cell proteins, at least about 135% more host-cell proteins, at least about 140% more host-cell proteins, at least about 145% more host-cell proteins, at least about 150% more host-cell proteins, at least about 155% more host-cell proteins, at least about 160% more host-cell proteins, at least about 165% more host-cell proteins, at least about 170% more host-cell proteins, at least about 175% more host-cell proteins, at least about 180% more host-cell proteins, at least about 185% more host-cell proteins, at least about 190% more host-cell proteins, at least about 195% more host-cell proteins, or at least about 200% more host-cell proteins. In one aspect, the chromatography support can be an affinity chromatography support.

In some exemplary embodiments, the method for characterizing host-cell proteins in a sample can comprise steps for enriching host-cell proteins in the sample by contacting the sample with a chromatography support and performing a fractionation step, wherein the method can be capable of characterizing at least about 20% more host-cell proteins than a method comprising the fractionation step and not the enriching step. In one aspect, the method can be capable of characterizing at least about 20% more host-cell proteins, at least about 25% more host-cell proteins, at least about 30% more host-cell proteins, at least about 35% more host-cell proteins, at least about 40% more host-cell proteins, at least about 45% more host-cell proteins, at least about 50% more host-cell proteins, at least about 55% more host-cell proteins, at least about 60% more host-cell proteins, at least about 65% more host-cell proteins, at least about 70% more host-cell proteins, at least about 75% more host-cell proteins, at least about 80% more host-cell proteins, at least about 85% more host-cell proteins, at least about 90% more host-cell proteins, at least about 95% more host-cell proteins, at least about 100% more host-cell proteins, at least about 105% more host-cell proteins, at least about 110% more host-cell proteins, at least about 115% more host-cell proteins, at least about 120% more host-cell proteins, at least about 125% more host-cell proteins, at least about 130% more host-cell proteins, at least about 135% more host-cell proteins, at least about 140% more host-cell proteins, at least about 145% more host-cell proteins, at least about 150% more host-cell proteins, at least about 155% more host-cell proteins, at least about 160% more host-cell proteins, at least about 165% more host-cell proteins, at least about 170% more host-cell proteins, at least about 175% more host-cell proteins, at least about 180% more host-cell proteins, at least about 185% more host-cell proteins, at least about 190% more host-cell proteins, at least about 195% more host-cell proteins, or at least about 200% more host-cell proteins. In one aspect, the chromatography support can be an affinity chromatography support.

In some exemplary embodiments, the method for characterizing host-cell proteins in a sample can comprise steps for enriching host-cell proteins in the sample by contacting the sample with a chromatography support and performing a fractionation step, wherein the method can be capable of characterizing about 20%-200% more host-cell proteins than a method comprising the enriching step and not the fractionation step. In one aspect, the method can be capable of characterizing about 20%-about 30% more host-cell proteins, about 30%-about 40% more host-cell proteins, about 40%-about 50% more host-cell proteins, about 50%-about 60% more host-cell proteins, about 60%-about 70% more host-cell proteins, about 70%-about 80% more host-cell proteins, about 80%-about 90% more host-cell proteins, about 90%-about 100% more host-cell proteins, about 100%-about 150% more host-cell proteins, or about 100%-about 200% more host-cell proteins. In one aspect, the chromatography support can be an affinity chromatography support.

In some exemplary embodiments, the method for characterizing host-cell proteins in a sample can comprise steps for enriching host-cell proteins in the sample by contacting the sample with a chromatography support and performing a fractionation step, wherein the method can be capable of characterizing about 20%-200% more host-cell proteins than a method comprising the fractionation step and not the enriching step. In one aspect, the method can be capable of characterizing about 20%-about 30% more host-cell proteins, about 30%-about 40% more host-cell proteins, about 40%-about 50% more host-cell proteins, about 50%-about 60% more host-cell proteins, about 60%-about 70% more host-cell proteins, about 70%-about 80% more host-cell proteins, about 80%-about 90% more host-cell proteins, about 90%-about 100% more host-cell proteins, about 100%-about 150% more host-cell proteins, or about 100%-about 200% more host-cell proteins. In one aspect, the chromatography support can be an affinity chromatography support.

In some exemplary embodiments, the method for characterizing a host-cell protein can comprise subjecting the sample matrix having host-cell proteins to non-denaturing digestion conditions to form a mixture and enriching host-cell proteins in said mixture by contacting the mixture with a chromatography support. The chromatography support can be a liquid chromatography support. As explained above, the liquid chromatography support can include reversed phase liquid chromatography, ion-exchange chromatography, size exclusion chromatography, affinity chromatography, mixed-mode chromatography, hydrophobic chromatography or mixed-mode chromatography.

As used herein, the “non-denaturing digestion conditions” or “native conditions” can include conditions that do not cause protein denaturation. Protein denaturing can refer to a process in which the three-dimensional shape of a molecule is changed from its native state without rupture of peptide bonds. The protein denaturation can be carried out using a protein denaturing agent, such as chaotropic agents. Chaotropic solutes increase the entropy of the system by interfering with intramolecular interactions mediated by non-covalent forces such as hydrogen bonds, van der Waals forces, and hydrophobic effects. Non-limiting examples for non-denaturing conditions include water or buffers. The water used can be distilled and/or deionized. In some exemplary embodiments, the solvents can be HPLC grade. Non-limiting examples of buffers can include ammonium acetate, tris-hydrochloride, ammonium bicarbonate, ammonium formate, or combinations thereof. In one aspect, the concentration of the buffer can be at most 1M.

In some exemplary embodiments, the method for characterizing a host-cell protein can comprise subjecting the sample matrix having host-cell proteins to non-denaturing digestion conditions to form a mixture and enriching host-cell proteins in said mixture by contacting the mixture with an affinity chromatography support.

In some exemplary embodiments, the method for characterizing a host-cell protein can comprise subjecting the sample matrix having host-cell proteins to non-denaturing digestion conditions to form a mixture and enriching host-cell proteins in said mixture by contacting the mixture with a protein A affinity chromatography support.

In some exemplary embodiments, the method for characterizing a host-cell protein can comprise subjecting the sample matrix having host-cell proteins to non-denaturing digestion conditions to form a mixture, enriching host-cell proteins in said mixture by contacting the mixture with a chromatography support and collecting the flow-through from the affinity chromatography support.

In some exemplary embodiments, the method for characterizing a host-cell protein can comprise subjecting the sample matrix having host-cell proteins to non-denaturing digestion conditions to form a mixture, enriching host-cell proteins in said mixture by contacting the mixture with a chromatography support and characterizing at least one of the host-cell proteins using a mass spectrometer. In one aspect, the mass spectrometer can be a tandem mass spectrometer. The tandem mass spectrometer can be tandem-in-space or tandem-in-time. In one aspect, the mass spectrometer can be coupled to a liquid chromatography system. In another aspect, the mass spectrometer can be coupled to a nano liquid chromatography system. In one aspect, the mobile phase used to elute the protein in liquid chromatography can be a mobile phase that can be compatible with a mass spectrometer. In a specific aspect, the mobile phase can be ammonium acetate, ammonium bicarbonate, or ammonium formate, acetonitrile, water, formic acid, a volatile acid, or combinations thereof. In one aspect, the chromatography support can be an affinity chromatography support. In one aspect, the method can further comprise using FAIMS device for characterization.

In some exemplary embodiments, the method for characterizing a host-cell protein can comprise subjecting the sample matrix having host-cell proteins to non-denaturing digestion conditions to form a mixture, enriching host-cell proteins in said mixture by contacting the mixture with a chromatography support, wherein the method can be capable of characterizing at least about 50% more host-cell proteins than a method comprising not comprising contacting the mixture with a chromatography support. In one aspect, the method can be capable of characterizing at least about 50% more host-cell proteins, at least about 75% more host-cell proteins, at least about 100% more host-cell proteins, at least about 125% more host-cell proteins, at least about 150% more host-cell proteins, at least about 175% more host-cell proteins, at least about 200% more host-cell proteins, at least about 225% more host-cell proteins, at least about 250% more host-cell proteins, at least about 275% more host-cell proteins, at least about 300% more host-cell proteins, at least about 325% more host-cell proteins, at least about 350% more host-cell proteins, at least about 375% more host-cell proteins, at least about 400% more host-cell proteins, at least about 425% more host-cell proteins, at least about 450% more host-cell proteins, at least about 475% more host-cell proteins, at least about 500% more host-cell proteins, at least about 525% more host-cell proteins, at least about 550% more host-cell proteins, at least about 575% more host-cell proteins, at least about 600% more host-cell proteins, at least about 625% more host-cell proteins, at least about 650% more host-cell proteins, at least about 675% more host-cell proteins, at least about 700% more host-cell proteins, at least about 725% more host-cell proteins, at least about 750% more host-cell proteins, at least about 775% more host-cell proteins, at least about 800% more host-cell proteins, at least about 825% more host-cell proteins, at least about 850% more host-cell proteins, at least about 875% more host-cell proteins, at least about 900% more host-cell proteins, at least about 925% more host-cell proteins, at least about 950% more host-cell proteins, at least about 975% more host-cell proteins, or at least about 1000% more host-cell proteins. In one aspect, the chromatography support can be an affinity chromatography support. In one aspect, the method can further comprise using FAIMS device for characterization.

In some exemplary embodiments, the method for characterizing a host-cell protein can comprise subjecting the sample matrix having host-cell proteins to non-denaturing digestion conditions to form a mixture, enriching host-cell proteins in said mixture by contacting the mixture with a chromatography support, wherein the method can be capable of characterizing about 50%-about 100% more host-cell proteins than a method not comprising contacting the mixture with an affinity chromatography support. In one aspect, the method can be capable of characterizing about 50%-about 100% more host-cell proteins, about 50%-about 500% more host-cell proteins, about 100%-about 500% more host-cell proteins, about 100%-about 1000% more host-cell proteins, or about 500%-about 1000% more host-cell proteins. In one aspect, the chromatography support can be an affinity chromatography support. In one aspect, the method can further comprise using FAIMS device for characterization.

In some exemplary embodiments, the method for characterizing a host-cell protein can comprise subjecting the sample matrix having host-cell proteins to non-denaturing digestion conditions to form a mixture, enriching host-cell proteins in said mixture by contacting the mixture with an affinity chromatography support and characterizing at least one of the host-cell proteins using a FAIMS.

In some exemplary embodiments, the method for characterizing a host-cell protein can comprise subjecting the sample matrix having host-cell proteins to non-denaturing digestion conditions to form a mixture, enriching host-cell proteins in said mixture by contacting the mixture with an affinity chromatography support and characterizing at least one of the host-cell proteins using FAIMS-MS. In one aspect, the carrier gas used for FAIMS device can include volatile chemical modifiers. In one aspect, the volatile chemical modifier can be isopropanol. In another aspect, FAIMS device can be used in conjunction with MS. In another specific aspect, FAIMS device can be used in conjunction with LC and MS. In another specific aspect, the FAIMS-MS can be coupled with LC.

In some exemplary embodiments, the method for characterizing a host-cell protein can comprise subjecting the sample matrix having host-cell proteins to non-denaturing digestion conditions to form a mixture, enriching host-cell proteins in said mixture by contacting the mixture with an affinity chromatography support and characterizing at least one of the host-cell proteins using nLC-FAIMS-MS.

In some exemplary embodiments, the method for characterizing a host-cell protein can comprise enriching host-cell proteins in the sample matrix by contacting the mixture with an affinity chromatography support to form a sample, subjecting the sample having host-cell proteins to non-denaturing digestion conditions to form a mixture, and characterizing at least one of the host-cell proteins using FAIMS, wherein the method can be capable of characterizing at least about 15% more host-cell proteins than a method not comprising FAIMS. In one aspect, the method can be capable of characterizing at least about 15% more host-cell proteins, at least about 16% more host-cell proteins, at least about 17% more host-cell proteins, at least about 18% more host-cell proteins, at least about 19% more host-cell proteins, at least about 20% more host-cell proteins, at least about 21% more host-cell proteins, at least about 22% more host-cell proteins, at least about 23% more host-cell proteins, at least about 24% more host-cell proteins, at least about 25% more host-cell proteins, at least about 26% more host-cell proteins, at least about 27% more host-cell proteins, at least about 28% more host-cell proteins, at least about 29% more host-cell proteins, at least about 30% more host-cell proteins, at least about 31% more host-cell proteins, at least about 32% more host-cell proteins, at least about 33% more host-cell proteins, at least about 34% more host-cell proteins, at least about 35% more host-cell proteins, at least about 36% more host-cell proteins, at least about 37% more host-cell proteins, at least about 38% more host-cell proteins, at least about 39% more host-cell proteins, at least about 40% more host-cell proteins, at least about 41% more host-cell proteins, at least about 42% more host-cell proteins, at least about 43% more host-cell proteins, at least about 44% more host-cell proteins, at least about 45% more host-cell proteins, at least about 46% more host-cell proteins, at least about 47% more host-cell proteins, at least about 48% more host-cell proteins, at least about 49% more host-cell proteins, or at least about 50% more host-cell proteins.

In some exemplary embodiments, the method for characterizing a host-cell protein can comprise enriching host-cell proteins in the sample matric by contacting the mixture with an affinity chromatography support to form a sample, subjecting the sample having the host-cell proteins to non-denaturing digestion conditions to form a mixture, and characterizing at least one of the host-cell proteins using FAIMS, wherein the method can be capable of characterizing at least about 15 to about 60% more host-cell proteins than a method not comprising FAIMS.

In some exemplary embodiments, a method for characterizing host-cell proteins in a sample matrix can comprise (a) enriching host-cell proteins in the sample by contacting the sample with an affinity chromatography support and (b) characterizing at least one of the host-cell proteins using FAIMS.

In some exemplary embodiments, a method for characterizing host-cell proteins in a sample matrix can comprise (a) enriching host-cell proteins in the sample by contacting the sample with an affinity chromatography support and (b) characterizing at least one of the host-cell proteins using FAIMS, wherein the method is capable of characterizing at least about 30% more host-cell proteins than a method not comprising step (b). In one aspect, the method can be capable of characterizing at least about 30% more host-cell proteins, at least about 35% more host-cell proteins, at least about 40% more host-cell proteins, at least about 45% more host-cell proteins, at least about 50% more host-cell proteins, at least about 55% more host-cell proteins, at least about 60% more host-cell proteins, at least about 65% more host-cell proteins, at least about 70% more host-cell proteins, at least about 75% more host-cell proteins, at least about 80% more host-cell proteins, at least about 85% more host-cell proteins, at least about 90% more host-cell proteins, at least about 95% more host-cell proteins, or at least about 100% more host-cell proteins.

In some exemplary embodiments, a method for characterizing host-cell proteins in a sample matrix can comprise (a) enriching host-cell proteins in the sample by contacting the sample with an affinity chromatography support and (b) characterizing at least one of the host-cell proteins using FAIMS, wherein the method is capable of characterizing at least about 30% to about 75% more host-cell proteins than a method not comprising step (b).

It is understood that the methods are not limited to any of the aforesaid protein, host-cell protein, chromatography support, mass spectrometry, fractionation method and that the methods for characterizing host-cell proteins may be conducted by any suitable means.

The consecutive labeling of method steps as provided herein with numbers and/or letters is not meant to limit the method or any embodiments thereof to the particular indicated order.

Various publications, including patents, patent applications, published patent applications, accession numbers, technical articles and scholarly articles are cited throughout the specification. Each of these cited references is incorporated by reference, in its entirety and for all purposes, herein.

The present invention will be more fully understood by reference to the following Examples. They should not, however, be construed as limiting the scope of the invention

### EXAMPLES

### (A) Number of HCPs Identified for a Preparation Comprising Ab1.

Materials. Deionized water was provided by a Milli-Q integral water purification system installed with MilliPak Express 20 filter (MilliporeSigma, Burlington, Mass.). Ammonium acetate (LC/MS grade), acetic acid and ammonium bicarbonate (LC/MS grade), Sequencing grade modified trypsin supplied with resuspension buffer from Promega, UltraPure 1M Tris-HCl pH 7.5 from Invitrogen, UltraPure 1M Tris-HCl pH 8 from Invitrogen, Trifluoroacetic Acid (TFA, Sequencing Grade) from Thermo Scientific, Acetonitrile (Optima LC/MS Grade) from Fisher, Glacial Acetic Acid from Sigma-Aldrich, Iodoacetamide from Sigma-Aldrich, Dithiothreitol (DTT) from Sigma-Aldrich, Urea (ultrapure) from Alfa Aesar, Dulbecco's phosphate-buffered saline (DPBS), pH 8.4 from Thermo Scientific, rProtein A Sepharose Fast Flow antibody purification resin from GE Healthcare, and Ammonium acetate (LC/MS grade) from Sigma-Aldrich. The preparation analyzed for HCPs comprised antibody Ab1.

Data analysis. Database search for peptides was performed using SEQUEST and MASCOT embedded into Proteome Discoverer 2.2 (Thermo Fisher Scientific) against the SwissProt mouse protein database. The search parameters were: 20 ppm tolerance for precursor ion masses, 0.5 Da tolerance for fragment ion masses analyzed by Ion Trap. Trypsin was specified during the database search. Methionine oxidation (+16 Da) was set as a variable modification. The false discovery rate (FDR) was determined by using the target-decoy strategy and was set to 1% for peptide identification and 5% for protein identification with a minimum of 1 unique peptides detected per protein (Alexander S. Hebert et al., The One Hour Yeast Proteome, 13 MOLECULAR & CELLULAR PROTEOMICS339-347 (2013)).

### Example 1

### HCP in Harvested Cell Culture Fluid

The Harvested Cell Culture Fluid was dried down and reconstitute in 8 M urea, 100 mM Tris-HCl. Samples were reduced with 10 mM dithiothreitol and incubated for 30 min at 50° C. The reduced samples were then alkylated with 15 mM iodoacetamide for 1 hour in the dark. Following alkylation, samples were buffer exchanged into 100 mM ammonium bicarbonate with molecular weight cutoff filters and digested with trypsin (1:20 w/w enzyme:substrate ratio) at 37° C. in the dark overnight The digestion was then stopped by addition of trifluoroacetic acid (TFA). The resulting tryptic peptides were then separated by reversed phase liquid chromatography followed by on-line mass spectrometry analysis. Separation was performed using a Thermo Scientific Easy-nLC 1200 by first concentrating and desalting the tryptic peptides on a Thermo Scientific Acclaim PepMap™ 100 trap column (C18, 75 μm ID, 2 cm bed length, 3 μm particle size, 100 Å pore size) and then separating them on a New Objective PicoFrit Column (360 μm OD, 75 μm ID, 10 μm tip ID, 25 cm bed length) packed with Acquity BEH stationary phase (C18, 1.7 μm particle size, 130 Å pore size) using water containing 0.1% formic acid (mobile phase A) and 80% acetonitrile/20% water containing 0.1% formic acid (mobile phase B). Peptides were separated using a gradient profile held at 6% mobile phase B for the first 10 minutes, then increased from 6% to 50% mobile phase B over the next 120 minutes. MS and MS/MS experiments were conducted on a Thermo Scientific Orbitrap Fusion Lumos Tribrid mass spectrometer with higher-energy collisional dissociation (HCD) employed for peptide fragmentation for MS/MS experiments (Thermo Orbitrap Fusion (Q-OT-qIT, Thermo Fisher Scientific, San Jose, Calif., USA)).

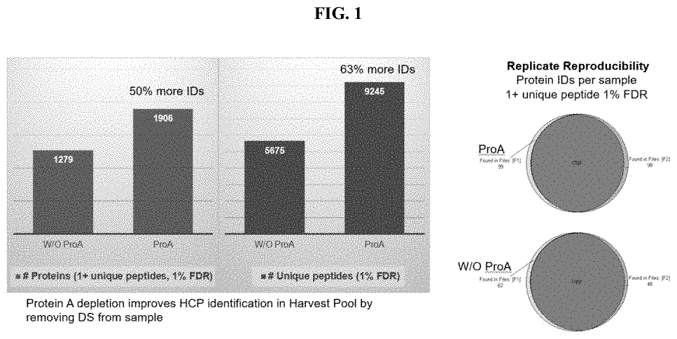

The number of HCPs identified in the HCCF without any treatment was 1279 (See FIG. 1). Further, the total number of unique peptides identified in the HCCF without any treatment was 5675.

### Example 2

### HCPs Characterized Using Protein A Depletion

### 2.1 Protein A Chromatography

Protein samples were dried down and resuspended in DPBS. rProtein A Sepharose was packed in columns and equilibrated with 5 column volumes of DPBS, pH 8.4. The protein sample was pipetted onto each column and incubated for 4 min at room temperature. The HCP flowthrough was collected and saved. Each column was washed with 3 column volumes of DPBS, pH 8.4 and the HCP eluate was combined with the flow-through. Collected HCP eluates were buffer exchanged into 50 mM ammonium acetate.

### 2.2 HCP Analysis

HCP eluate was treated before analyzing them as illustrated in example 1.

The number of HCPs identified using protein A chromatography was 1906 (See FIG. 1). Further, the total number of unique peptides identified in the HCCF using protein A chromatography were 9245.

### Example 3

### HCPs Characterized Using a Fractionation Method

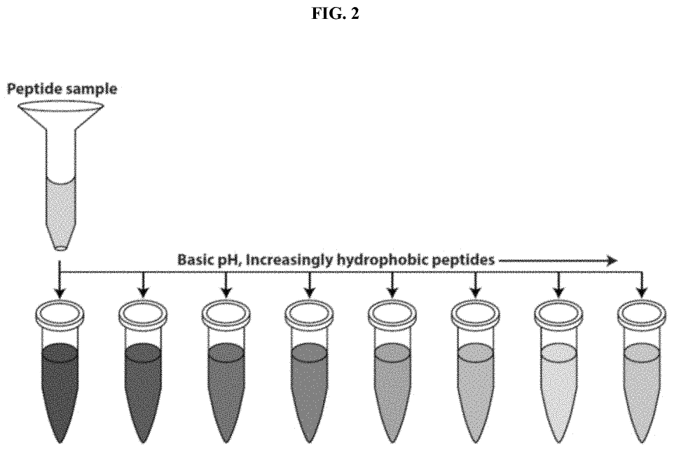

Pierce™ High pH Reversed-Phase Peptide Fractionation Kit was used for this step (See FIG. 2).

### 3.1 Conditioning of the Spin Columns

The protective white tip from the bottom of the column was removed and discarded and the column was placed into 2.0 mL sample tube. The tube was centrifuged at 5000×g for 2 minutes to remove the solution and pack the resin material and the liquid was discarded. The top screw cap was removed, and the column was loaded with 3004, of ACN (Fisher) into the column and the cap was replaced and the spin column was placed back into a 2.0 mL sample tube and centrifuged at 5000×g for 2 minutes. The ACN was discarded and the wash step was reported. The spin column was then washed twice with twice with 0.1% TFA solution (Thermo Scientific), as described above for the ACN wash.

### 3.2 Fractionation of the HCCF

The elution solutions were prepared as shown according to Table 1. 100 μg of protein from the Harvested Cell Culture Fluid was added in 300 μL of 0.1% TFA solution. The spin column was placed into a new 2.0 mL sample tube and the sample solution was loaded onto the column. After replacing the top cap, the sample tube was centrifuged at 3000×g for 2 minutes. The “flow-through” fraction was collected. The column was then placed into a new 2.0 mL sample tube and [300] μL of water was added onto the column and centrifuged again to collect the “wash” fraction. The column was then placed into a new 2.0 mL sample tube and [300] μL of the appropriate elution solution was added to it and centrifuged at 3000×g for 2 minutes to collect the fraction. This step was repeated for the remaining step gradient fractions using the appropriate elution solutions from Table 1 in new 2.0 mL sample tubes. The liquid contents were evaporated for each sample tube to dryness using vacuum centrifugation (e.g., SpeedVac concentrator). The dry samples were re-suspended in an appropriate volume of 0.1% formic acid (FA) before LC-MS analysis.

### 3.3 HCP Analysis

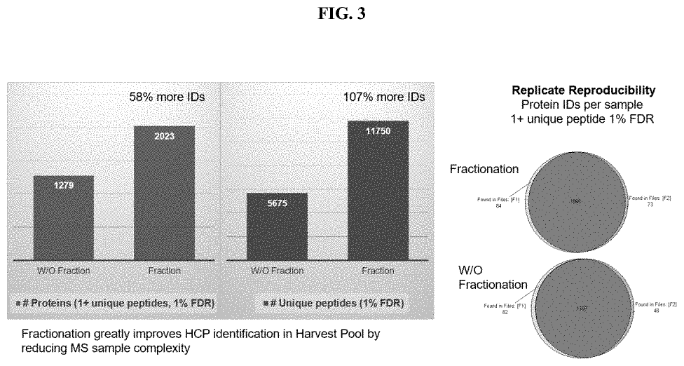

Each of the fractions were treated before analyzing as illustrated in example 1. The number of HCPs identified by the fractionation method was 2023 (See FIG. 3). Further, the total number of unique peptides identified in the HCCF using the fractionation method was 11750.

TABLE 1

| Fraction | Acetonitrile | Acetonitrile | Triethylamine |
|---|---|---|---|
| No. | (%) | (μL) | (0.1%) (μL) |
| 1 | 5.0 | 50 | 950 |
| 2 | 7.5 | 75 | 926 |
| 3 | 10.0 | 100 | 900 |
| 4 | 12.5 | 125 | 875 |
| 5 | 15.0 | 150 | 850 |
| 6 | 17.5 | 175 | 825 |
| 7 | 20.0 | 200 | 800 |
| 8 | 50.0 | 500 | 500 |

### Example 4

### HCPs Characterized Using Protein A Depletion and a Fractionation Method

### 4.1 Protein A Chromatography

Protein A chromatography was performed using the method as described in Example 2.

The proteins in the flow-through were reduced with 10 mM dithiothreitol and incubated for 30 min at 50° C. The reduced samples were then alkylated with 15 mM iodoacetamide for 1 hour in the dark. Following alkylation, samples were buffer exchanged into 100 mM ammonium bicarbonate with molecular weight cutoff filters and digested with trypsin (1:20 w/w enzyme:substrate ratio) at 37° C. in the dark overnight The digestion was then stopped by addition of trifluoroacetic acid (TFA). The resulting tryptic peptides were then subjected to the fractionation step.

### 4.2 Fractionation Step

The resulting tryptic peptides were fractionated as described in Example 3.

### 4.3 HCP Analysis

The fractionated peptides obtained from step 4.2 were each subjected to separation by using reversed phase liquid chromatography followed by on-line mass spectrometry analysis. Separation was performed using a Thermo Scientific Easy-nLC 1200 by first concentrating and desalting the tryptic peptides on a Thermo Scientific Acclaim PepMap™ 100 trap column (C18, 75 μm ID, 2 cm bed length, 3 μm particle size, 100 Å pore size) and then separating them on a New Objective PicoFrit Column (360 μm OD, 75 μm ID, 10 μm tip ID, 25 cm bed length) packed with Acquity BEH stationary phase (C18, 1.7 μm particle size, 130 Å pore size) using water containing 0.1% formic acid (mobile phase A) and 80% acetonitrile/20% water containing 0.1% formic acid (mobile phase B). Peptides were separated using a gradient profile held at 6% mobile phase B for the first 10 minutes, then increased from 6% to 50% mobile phase B over the next 120 minutes. MS and MS/MS experiments were conducted on a Thermo Scientific Orbitrap Fusion Lumos Tribrid mass spectrometer with higher-energy collisional dissociation (HCD) employed for peptide fragmentation for MS/MS experiments (Thermo Orbitrap Fusion (Q-OT-qIT, Thermo Fisher Scientific, San Jose, Calif., USA)).

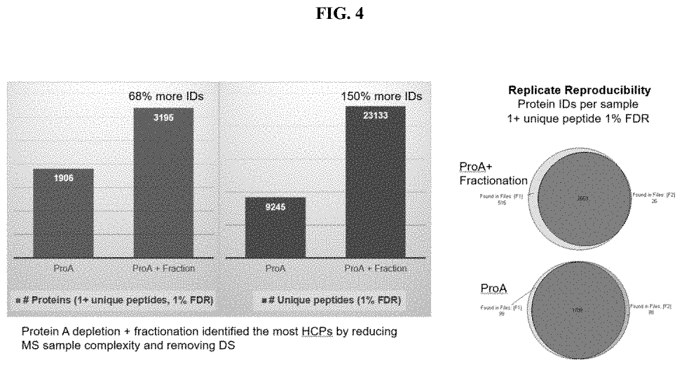

The number of HCPs identified by the method (with protein A and fractionation steps) was 3195 (See FIG. 4). Further, the total number of unique peptides identified in the HCCF using the modified method (with protein A and fractionation steps) was23133.

### Example 5

### HCPs Characterized Using Normal Digestion

### 5.1 Sample Preparation

Ab1 was digested with trypsin added to the mixture to substrate concentration of 1:20 in 100 mM ammonium bicarbonate, pH 7.4.

### 5.2 HCP Analysis

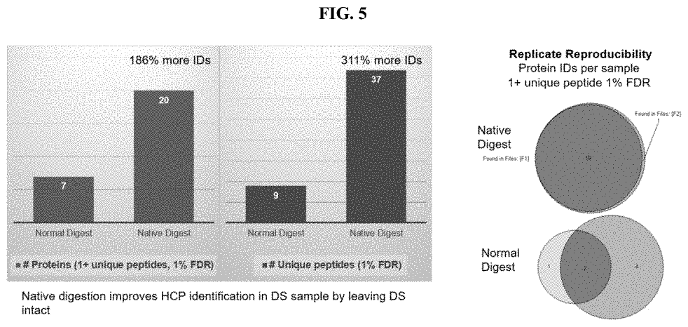

The digests obtained were analyzed for HCPs using the method outlined in example 1. The number of HCPs identified by the method was 7 (See FIG. 5) and the total number of unique peptides identified was 9.

### Example 6

### HCPs Characterized Using Native Digestion

### 6.1 Sample Preparation

Ab 1 was treated by drying the samples down and resuspending in 25 mM tris-HCl buffer, pH 8. Samples were digested with trypsin (1:400 w/w enzyme: substrate ratio) at 37° C. overnight with a final pH ˜7.4. Samples were reduced with 3 mM dithiothreitol and incubate for 10 min at 90° C. Samples were acidified to ˜0.2% formic acid and centrifuged at 15000×g for 2 min. The supernatant was used for LC/MS analysis.

### 6.2 HCP Analysis

The digests obtained were analyzed for HCPs using the method outlined in example 1. The number of HCPs identified by the method was 20 (See FIG. 5) and the total number of unique peptides identified was 37.

### Example 7

### HCPs Characterized Using Protein A Depletion Followed by Native Digestion

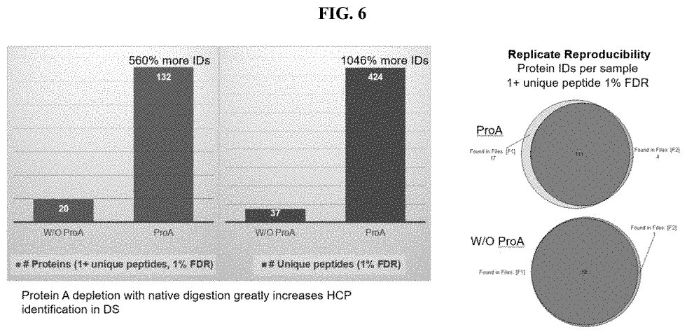

The digests from the experiment 6.1 were generated after Protein A chromatography depletion using the method as described in Example 2. The flow-through collected and analyzed as described above. The number of HCPs identified by this method (native digestion and protein A chromatography) was 132 (See FIG. 6) and the total number of unique peptides identified was 424.

### Example 8

### HCPs Characterized Using Protein A Depletion

### 8.1 Protein A Chromatography

Protein samples were dried down and resuspended in DPBS. rProtein A Sepharose was packed in columns and equilibrated with 5 column volumes of DPBS, pH 8.4. The protein sample was pipetted onto each column and incubated for 4 min at room temperature. The HCP flowthrough was collected and saved. Each column was washed with 3 column volumes of DPBS, pH 8.4 and the HCP eluate was combined with the flowthrough. Collected HCP eluates were buffer exchanged into 50 mM ammonium acetate.

### 8.2 HCP Analysis

The HCP eluate was dried down and reconstituted in 8 M urea, 100 mM Tris-HCl. Samples were reduced with 10 mM dithiothreitol and incubated for 30 min at 50° C. The reduced samples were then alkylated with 15 mM iodoacetamide for 1 hour in the dark. Following alkylation, samples were buffer exchanged into 100 mM ammonium bicarbonate with molecular weight cutoff filters and digested with trypsin (1:20 w/w enzyme:substrate ratio) at 37° C. in the dark overnight The digestion was then stopped by addition of trifluoroacetic acid (TFA). The resulting tryptic peptides were then separated by reversed phase liquid chromatography followed by on-line mass spectrometry analysis. Separation was performed using a Thermo Scientific Easy-nLC 1200 by first concentrating and desalting the tryptic peptides on a Thermo Scientific Acclaim PepMap™ 100 trap column (C18, 75 μm ID, 2 cm bed length, 3 μm particle size, 100 Å pore size) and then separating them on a New Objective PicoFrit Column (360 μm OD, 75 μm ID, 10 μm tip ID, 25 cm bed length) packed with Acquity BEH stationary phase (C18, 1.7 μm particle size, 130 Å pore size) using water containing 0.1% formic acid (mobile phase A) and 80% acetonitrile/20% water containing 0.1% formic acid (mobile phase B). Peptides were separated using a gradient profile held at 6% mobile phase B for the first 10 minutes, then increased from 6% to 50% mobile phase B over the next 120 minutes. MS and MS/MS experiments were conducted on a Thermo Scientific Orbitrap Fusion Lumos Tribrid mass spectrometer with higher-energy collisional dissociation (HCD) employed for peptide fragmentation for MS/MS experiments (Thermo Orbitrap Fusion (Q-OT-qIT, Thermo Fisher Scientific, San Jose, Calif., USA)).

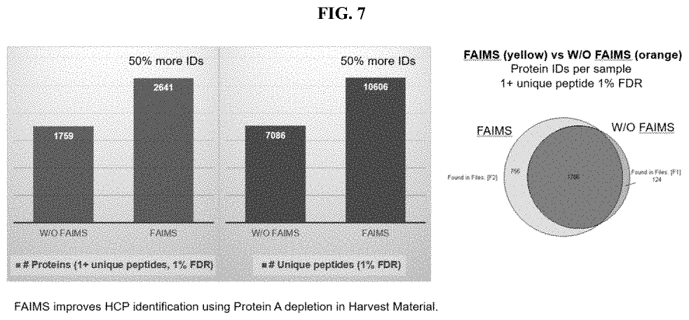

The number of HCPs of Ab1 in the HCCF identified using protein A chromatography was 1759 (See FIG. 7) and the total number of unique peptides was 7086.

### Example 9

### HCPs Characterized Using Protein A Depletion and FAIMS Device

The proteins in the flow-through from the protein A chromatography carried out in example 8 were digested to peptides and analyzed using system as described below.

For FAIMS-enabled experiment the settings were identical as described in example above except the FAIMS device was placed between the nanoelectrospray source and the mass spectrometer. FAIMS separations were performed with the following settings: inner electrode temperature=100° C. (except where noted), outer electrode temperature=100° C., FAIMS carrier gas flow =4.6 L/min, asymmetric waveform with DV=−5000 V, entrance plate voltage=250 V, and CV settling time=25 ms. The FAIMS carrier gas is N₂ only, and the ion separation gap is 1.5 mm. The noted CVs were applied to the FAIMS electrodes. For external stepping or single CV experiments the selected CV was applied to all scans throughout the analysis. For internal CV stepping experiments, each of the selected CVs was applied to sequential survey scans and MS/MS cycles (1 s); the MS/MS CV was always paired with the appropriate CV from the corresponding survey scan.

The number of HCPs of Ab 1 in the HCCF identified using protein A chromatography in conjunction with use of FAIMS device was 2641 (See FIG. 7) and the total number of unique peptides was 10606.

### Example 10

### HCPs Characterized Using Native Digestion and Protein A Chromatography

### 10.1 Protein A Chromatography

Ab 1 sample was purified using a Protein A chromatography depletion using the method as described in Example 2. The flow-through was collected and digested as described below.

### 10.2 Native Digestion

Ab 1 was treated by drying the samples down and resuspending in 25 mM tris-HCl buffer, pH 8. Samples were digested with trypsin (1:400 w/w enzyme: substrate ratio) at 37° C. overnight with a final pH ˜7.4. Samples were reduced with 3 mM dithiothreitol and incubate for 10 min at 90° C. Samples were acidified to ˜0.2% formic acid and centrifuged at 15000×g for 2 min. Supernatant was used for LC/MS analysis.

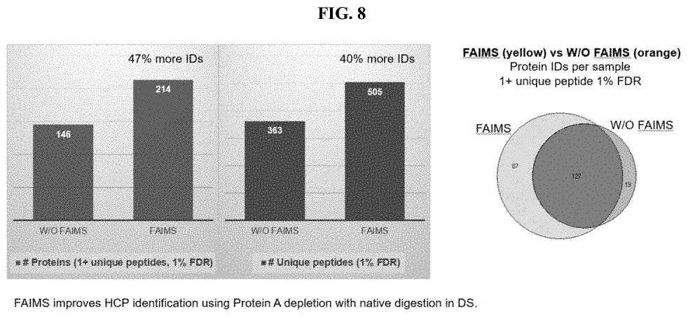

The number of HCPs of mAb1 identified by this method (native digestion and protein A chromatography) was 146 (See FIG. 8) and the total number of unique peptides identified was 363.

### Example 11

### HCPs Characterized Using Native Digestion, Protein A Chromatography and FAIMS

The supernatant from example 10 was also analyzed using a FAIMS device as described in example 9.

The number of HCPs identified using the method using native digestion conditions after in conjunction with use of FAIMS device was 214 (See FIG. 8) and the total number of unique peptides was 505.

(B) Number of HCPs Identified for a Preparation Comprising Ab1.

Chemicals. Glacial acetic acid, urea, iodoacetamide (IAM), and dithiothreitol (DTT) were purchased from Sigma-Aldrich (St. Louis, Mo.). Trifluoroacetic acid (TFA), Formic acid (FA), acetonitrile, and Dulbecco's phosphate-buffered saline (DPBS 10×, no calcium, no magnesium) were obtained from Thermo Fisher Scientific (Rockford, Ill.) while rProtein A Sepharose Fast Flow beads were purchased from GE Healthcare (Uppsala, Sweden). Sequencing grade modified trypsin with resuspension buffer was procured from Promega (Madison, Wis.), tris-HCl buffer (pH 7.5 and 8.0) was obtained from Invitrogen (Carlsbad, Calif.), and humanized IgG1κ monoclonal antibody standard RM 8671 was purchased from the National Institute of Standards and Technology (NIST).

Protein A Depletion. Drug substance was buffer exchanged into DPBS, adjusted to pH 8.4. 1 mL protein A columns were equilibrated with five column volumes of DPBS. Drug substance was added to the protein A column and incubated for 4 min at room temperature. Each column was washed with three column volumes of DPBS and the eluate and flow-through were collected. Flow-through and eluate were buffer exchanged into 50 mM ammonium acetate with Amicon Ultra 3 kDa centrifugal filters (Millipore) by centrifuging at 3000 g and 5° C. The protein concentration was measured using a NanoDrop 1000 spectrophotometer (Thermo Fisher Scientific). Protein from each sample was dried in vacuo or stored at −80° C.

Standard Digestion. Dried drug substance samples were reconstituted in 8 M urea/100 mM Tris-HCl. The protein was reduced with 10 mM DTT and incubated for 30 min at 50° C. Samples were cooled to room temperature and alkylated with 15 mM IAM for 1 hour in the dark. The mixture was buffer exchanged into 100 mM ammonium bicarbonate with Amicon Ultra 3 kDa centrifugal filters (Millipore) following manufacturer's instructions. Proteolytic digestion was performed with trypsin (1:20 trypsin:substrate ratio) overnight at 37° C. The digestion was quenched by acidifying to 0.2% FA.

Native Digestion. A detailed description of the native digestion is provided by Huang et al. 2017, supra. Briefly, samples were dried down and resuspended in 25 mM tris-HCl buffer, pH 8. Samples were then digested with trypsin (1:400 w/w enzyme:substrate ratio) at 37° C. overnight with a final pH of ˜7.4. Subsequently, samples were reduced with 3 mM DTT and incubate for 10 min at 90° C. Samples were acidified to ˜0.2% FA and centrifuged at 15000 g for 2 min. Supernatant was removed and used for LC-M5² analysis.

NanoLC-M5². Approximately 1 μg of digested protein was injected onto a C18 column (New Objective PicoFrit Column, 360 μm OD, 75 μm ID, 10 μm tip ID, 25 cm bed length packed with BEH C18 particles [1.7 μm, 130 Å, Waters]) with an Easy-nLC (Thermo Fisher Scientific). Mobile phase A contained 0.1% FA in water and mobile phase B contained 0.1% FA in 80% acetonitrile/20% water. The linear LC gradient was set up as follows: 0-10 min: 6% B, 130 min: 50% B, 140-155 min: 100% B. Eluant was analyzed on an Orbitrap Fusion Lumos Tribrid mass spectrometer equipped with a FAIMS Pro interface (Thermo Fisher Scientific). As the protein A depletion and native digest reduce the dynamic range of the peptides in the sample, we implemented standard proteomics MS settings (as an example, see Alexander S. Hebert et al., The One Hour Yeast Proteome, 13 MOLECULAR & CELLULAR PROTEOMICS 339-347 (2013)). Briefly, a survey scan was performed in the Orbitrap with a cycle time of one second. MS scans had an m/z range of 360-1600, a resolution of 60K, an AGC target of 5E5, and a maximum injection time of 50 ms. HCD fragmentation was performed between MS cycles with normalized collision energy of 30%, followed by analysis in the ion trap. MS² scans had a m/z range of 100-2000, an AGC target of 1E4, and maximum injection time of 35 ms. Dynamic exclusion duration was set to 30 seconds with a single repeat count and only precursors with charge states of +2 to +8 were analyzed. Operation of the FAIMS Pro interface has been described by Hebert et al. (Alexander S. Hebert et al., Comprehensive Single-Shot Proteomics with FAIMS on a Hybrid Orbitrap Mass Spectrometer, 90 ANALYTICAL CHEMISTRY 9529-9537 (2018)). Briefly, the FAIMS electrode temperatures were set to 100° C., FAIMS carrier gas flow was 4.7 L/min N_{2,} asymmetric waveform with DV was −5000 V, entrance plate voltage was 250 V, CV settling time was 25 ms, and CVs were set to −50 V, −65 V, and −85 V. When not used for an experiment, the FAIMS Pro interface was removed from the MS

Data Analysis. Database searches for peptide and protein identification were performed using SEQUEST and Mascot embedded into Proteome Discoverer 2.2 (Thermo Fisher Scientific) against the SwissProt mouse protein database, which included common contaminants. Mass tolerances were 20 ppm for precursor ion masses analyzed by the Orbitrap and 0.5 Da for fragment ion masses analyzed by the ion trap. Trypsin was specified during the database search. Methionine oxidation (+16 Da) was set as a variable modification and cysteine carbamidomethylation was set as a fixed modification for the normal (alkylated) digests. The false discovery rate (FDR) was determined by using the target-decoy strategy and was set to 1% for peptide identification and 5% for protein identification with a minimum of 2 unique peptides detected per protein.

One major challenge to HCP analysis by LC-M5² is the very low concentration of HCPs compared to the therapeutic protein in drug solution (DS) (˜1-100 ng HCP/mg product). Since tryptic peptides behave virtually the same in the mass spectrometer regardless of the protein they are processed from and the therapeutic protein is overwhelmingly abundant in DS, tryptic peptides from HCPs suffer from signal suppression and increased background during a typical analysis. To detect a 1 ppm HCP contaminant coeluting with a therapeutic protein in DS, the mass spectrometer requires a dynamic range over six orders of magnitude, which is beyond what current mass spectrometers can achieve. This sensitivity requirement necessitates the optimization of every step in a typical proteomics workflow, including digestion of the sample, chromatography/separation, and instrument analysis. However, as increased sensitivity is only a single facet of the analysis, one must also consider resources and sample throughput. Several methods for improving HCP detection were compared to make recommendations to achieve a balance between complexity, speed, and depth of analysis. To generate a broadly comparable dataset while optimizing HCP protocol, the National Institute of Standards & Technology Humanized IgG1κ Monoclonal Antibody standard (NISTmAb) was used, but results showed the same trends with other therapeutic proteins tested as well (data not shown).

### Example 12

### Method Comparison and Optimization Using NISTmAb

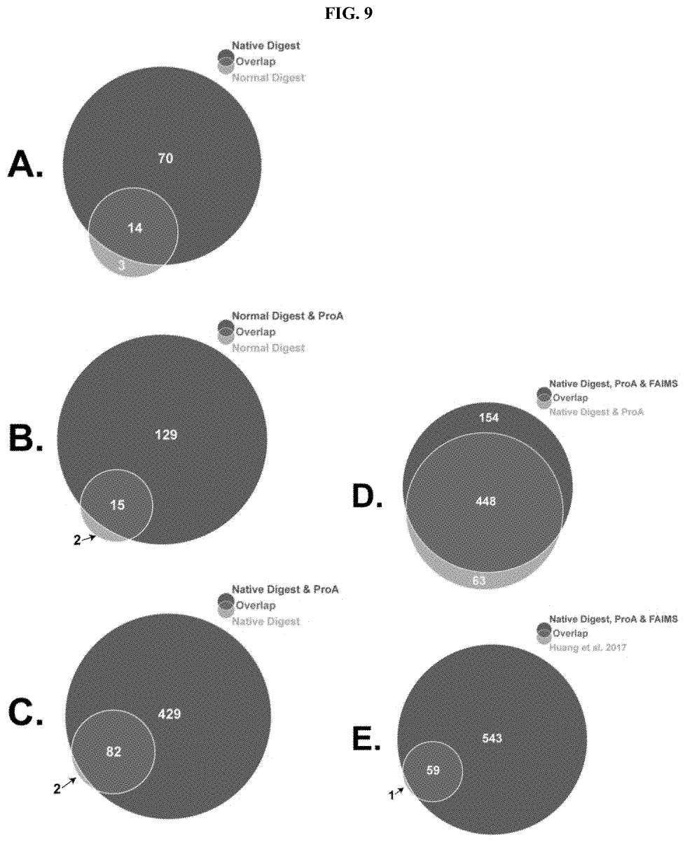

Previously, a simple and efficient method of alleviating the dynamic range issue in HCP analysis, coined the ‘native digest,’ was reported by Huang et al., supra. By selectively digesting lower abundance HCPs and leaving the relatively large and stable antibody intact, they dramatically reduced the interference from antibody peptides and, accordingly, detected a far greater number of HCPs than in a typical digest. Our findings corroborate these results (FIG. 9A); over four times the HCPs were identified than using the ‘native’ digest compared to a ‘normal’ tryptic digest (i.e. one with reduction and alkylation prior to digestion) as well as a proportional increase in the number of unique peptides (Table 1). For comparison, we depleted an antibody sample using a protein A column (FIG. 9B). Protein A depletion was found to be a more effective strategy than the native digest, detecting roughly ten times as many HCPs compared to the control sample (normal digest) as well as a proportional increase in the number of unique peptides. However, there are advantages to both procedures. For example, the native digest requires less sample preparation and starting material than the protein A protocol, which requires both depletion and digestion, but it should also be noted that the protein A depletion can be automated and the sample analysis does not require any additional instrument time.

TABLE 1
Number of HCPs and unique peptides detected in the analyses

| | # of Unique | # of HCPs |
|---|---|---|
| Sample Name | Peptides | Identified |
| Native Digest, ProA & FAIMS | 2838 | 602 |
| Native Digest & ProA | 2570 | 511 |
| Normal Digest, ProA & FAIMS | 660 | 185 |
| Normal Digest & ProA | 617 | 144 |
| Native Digest & FAIMS | 499 | 135 |
| Native Digest | 344 | 84 |
| Normal Digest & FAIMS | 55 | 20 |
| Normal Digest | 54 | 17 |

While these results represent a considerable improvement over traditional shotgun proteomics analysis of HCPs, most of the peptides detected in the above experiments were still from the antibody rather than the proteins of interest. Therefore, native digestion and protein A depletion in combination was tested to determine if one can further improve HCP peptide signal by decreasing interference from the DS compared to protein A depletion or native digestion independently. Indeed, comparing the native digest to the native digest after protein A depletion demonstrates a remarkable increase in sensitivity beyond what the already effective single methods can provide (FIG. 9C). From the combination of protein A depletion and native digestion, 511 HCPs in a purified DS sample were detected, far more than in the native digest sample (84 HCPs identified) and the protein A depletion with normal digest sample (144 HCPs identified). Furthermore, the results are highly complementary, with more than 99% of HCPs detected in previous NISTmAb analyses also being detected in the protein A depleted native digest sample. This indicates little to no bias in protein identifications between these depletion and digestion strategies. Given the frequency with which HCPs are copurified with the therapeutic protein (see Aboulaich et al., supra; Levy et al. (2014), supra; and Levy et al;. (2018), supra), there exists a reasonable concern that protein A depletion, or any other method that depletes the therapeutic protein or enriches the HCPs, could unintentionally remove HCPs as well. However, since virtually all HCPs detected in the analyses without protein A depletion were also detected in the protein A depleted samples, the gain in sensitivity more than outweighs any loss is due to interactions between the HCPs and protein A or therapeutic protein.

These results emphasize the importance of reducing background interference from the DS. After combining multiple depletion methods, however, we found that further removal of DS was no longer helpful as the major peaks detected in the protein A depleted native digest samples were no longer solely from DS peptides, but a combination of HCP, protein A, trypsin autolysis and other peptides. In essence, the dynamic range between the most abundant and least abundant peptides was reduced to roughly two orders of magnitude, and thus further removal of the therapeutic protein is unlikely to increase sensitivity to HCPs. However, this analysis also shows how complex purified DS samples are once the therapeutic protein has been depleted, with more than 500 proteins and thousands of peptides detected in a single sample (Table 1). Therefore, it seems likely that better separation of the peptides and faster MS analysis will be helpful to further increase analytical depth. Indeed, this strategy will likely increase protein identification in most shotgun proteomics analyses. However, it is important to consider the added complexity of such strategies. The use of fractionation for HCP analysis, for example, has been discussed previously (Kufer et al, supra). While it was found that fractionation of the DS was still helpful, it represents a considerable increase in instrument time, which is problematic for routine analyses. Ion mobility is an alternative that can achieve similar benefits to fractionation without the added sample preparation and instrument time (See Doneanu et al. (2015), supra).

### Example 13

### Method Comparison and Optimization Using NISTmAb

High-field asymmetric waveform ion mobility spectrometry (FAIMS) was investigated as a possible technique that could be used in lieu of fractionation of DS peptides. An overview of FAIMS has been reported in detail elsewhere (see Hebert et al. (2018), supra). Briefly, the FAIMS cell resides at the interface between the nanospray emitter and mass spectrometer transfer tube; inside is a circular electrode to which compensation voltages (CVs) can be applied. This enables gas-phase separation of ions before they enter the mass spectrometer. Since the CV can be changed rapidly (˜25 milliseconds/transition), it is possible to switch between multiple CVs throughout a single run and thereby simplify individual scans.

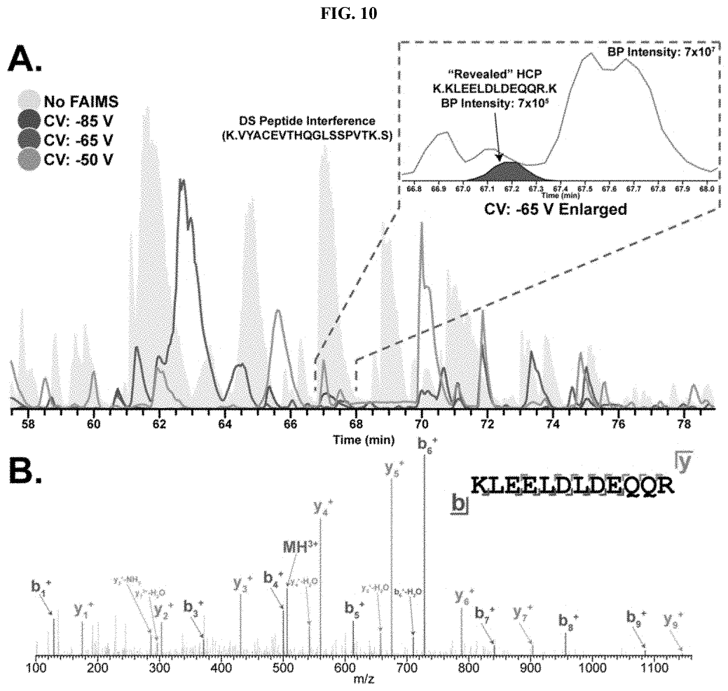

As an example, considering the same sample run with and without FAIMS, as shown in FIG. 10. Even for samples that are depleted of the therapeutic protein, such as by a protein A column and native digestion, many of the major peptides detected are still from the therapeutic protein. The full MS scan during the elution of these peptides indicated several ions that are of potential interest. While the main DS peptide ion is abundant, there are several other lower abundance ions which could be HCP peptides and warrant analysis but may not be selected for MS² fragmentation before the next peaks begin eluting. This is of particular concern since the HCP ions are often two orders of magnitude less intense than the DS peptide ions even in samples where the therapeutic protein has been depleted. By contrast, if the same sample is run using the FAIMS cell with three different CVs, three unique base peak chromatograms (BPC) were obtained. At a CV of −50 V, the BPC is similar to the sample run without FAIMS, and in the full MS spectrum, the same major DS peptide ion is observed. In the spectra with CVs of −85 and −65 V, the DS peptide ion disappears, enabling analysis of HCP peptides that were not detected without FAIMS. In this example, FAIMS essentially fractionates the sample into three different runs, without any major increase in duty cycle or additional sample preparation steps.

The primary advantage of FAIMS for HCP analysis is its ability to reduce sample complexity, allowing for detection of more low abundance peptides. In principle, it is analogous to other types of fractionation without requiring additional sample preparation or instrument time. Additionally, reduction of background noise due to the filtering effect of FAIMS can also allow for better precursor ion selection and improved MS² spectra, increasing confidence in peptide IDs. While the FAIMS interface can potentially reduce signal, any reduction in signal is accompanied by a decrease in background noise (i.e. the signal to noise ratio is improved in most observed cases). The addition of FAIMS was found to increase identification of HCPs by ˜20% compared to samples run without FAIMS (Table 1 and FIG. 9D).

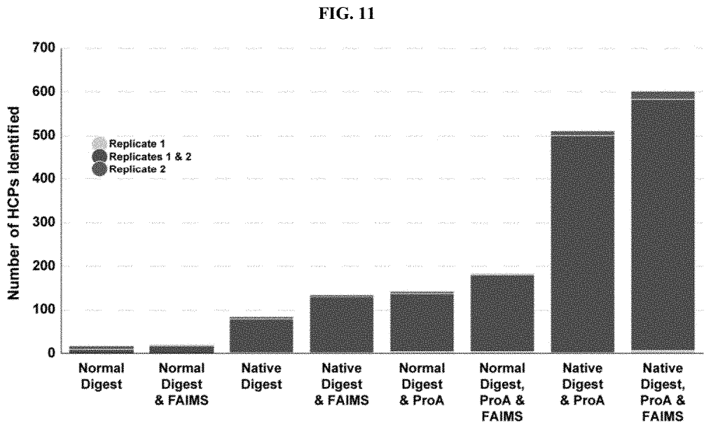

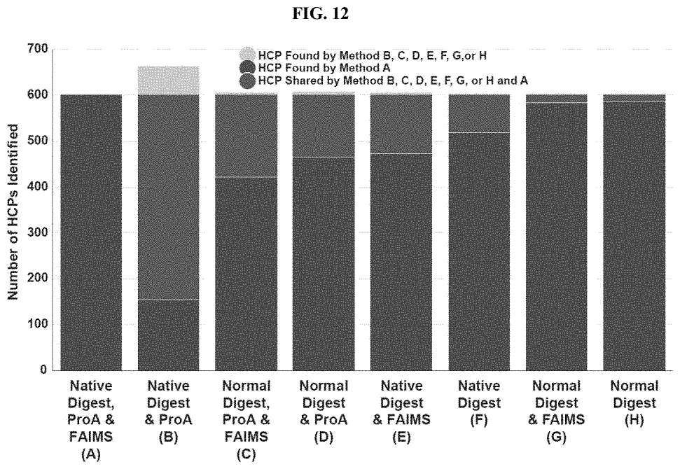

With this optimized workflow, not only a large number of HCPs (602 in the protein A depleted native digest sample using FAIMS) were identified, but it was found to be highly robust and agrees well with previously reported methods. Reproducibility is especially important when it comes to analyzing DS for HCP content, and we found that techniques described here are quite reproducible (FIG. 11). For example, HCP identifications in the optimized method described above vary by at most 4% between replicate samples. There was remarkably broad overlap even between different techniques or sample preparations, as shown by FIG. 12. The 63 proteins unique to the protein A depleted native digest sample detected without using FAIMS illustrate that the maximum number of HCPs can be obtained by combining the identifications obtained after running the sample once with FAIMS and once without FAIMS. These results also match well with those reported in literature, identifying 59 of the 60 proteins reported by Huang et al., supra using our optimized protocol (FIG. 9E). This consistency between replicates and protocols provides strong support for the use of these techniques in the routine analysis of HCPs during production of biopharmaceuticals.

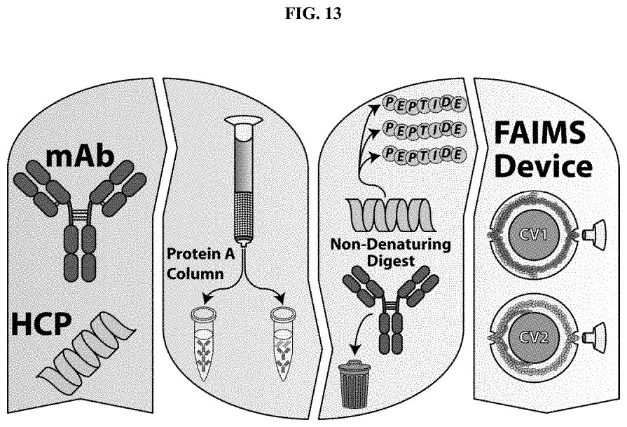

This multifactorial approach (shown in FIG. 13) that first depletes the sample of antibody on a protein A column, then specifically digests HCPs while precipitating any remaining antibody, and finally reduces spectral complexity through shotgun proteomics and compensation voltage (CV) switching using high-field asymmetric waveform ion mobility spectrometry (FAIMS) allowed for an order of magnitude greater analytical depth than any single method while maintaining the simplicity and high-throughput required for routine analysis of HCPs.

What is claimed is:

1. A method for characterizing host-cell proteins in a sample matrix, comprising:
enriching host-cell proteins in the sample matrix by contacting the sample matrix with an affinity chromatography support;
performing fractionation on a flowthrough from the affinity chromatography; and
characterizing at least one of the host-cell proteins using a mass spectrometer.

2. The method of claim 1, wherein the affinity chromatography support is protein A chromatography support.

3. The method of claim 1 further comprising washing the affinity chromatography support with a wash buffer and collecting the flow-through.

4. The method of claim 1, wherein the affinity chromatography support comprises protein A or protein G.

5. The method of claim 4, wherein the protein A or the protein G is immobilized on agarose or sepharose resin.

6. The method of claim 1, wherein the mass spectrometer is a tandem mass spectrometer. The method of claim 6, wherein the mass spectrometer is coupled with a liquid chromatography system.

8. The method of claim 7, wherein the liquid chromatography system is a nano-liquid chromatography system.

9. The method of claim 1 further comprising characterizing at least one of the host-cell proteins using High-Field Asymmetric Waveform Ion Mobility Spectrometry device.

10. The method of claim 1, wherein the sample matrix further comprises a protein of interest.

11. The method of claim 10, wherein the protein of interest is an antibody.

12. The method of claim 10, wherein the protein of interest is a fusion protein.

13. The method of claim 1, wherein the fractionation is a size-based fractionation.

14. The method of claim 1, wherein the fractionation is a hydrophobicity-based fractionation.

15. The method of claim 1, wherein fractionation is a charge-based fractionation.

16. The method of claim 1, wherein fractionation is a pI-based fractionation.

17. The method of claim 1, wherein the fractionation comprises fractionation by liquid chromatography.

18. The method of claim 17, wherein the liquid chromatography is reversed phase liquid chromatography.

19. The method of claim 1, wherein the method is capable of characterizing at least about 50% more host-cell proteins than a method that enriches host-cell proteins in the sample matrix by contacting the sample matrix with an affinity chromatography support without performing the fractionation step.

20. The method of claim 1, wherein the method is capable of characterizing at least about 50% more host-cell proteins than a method that performs a fractionation without enriching host-cell proteins in the sample matrix by contacting the sample matrix with an affinity chromatography support.

21. The method of claim 1, wherein the method is capable of characterizing at least about 50% to about 75% more host-cell proteins than a method that enriches host-cell proteins in the sample matrix by contacting the sample matrix with an affinity chromatography support without performing the fractionation step.

22. The method of claim 1, wherein the method is capable of characterizing at least about 50% to about 75% more host-cell proteins than a method that performs a fractionation without enriching host-cell proteins in the sample matrix by contacting the sample matrix with an affinity chromatography support.

23. A method for characterizing host-cell proteins in a sample matrix having a protein of interest, comprising:
enriching host-cell proteins in the sample matrix by contacting the sample matrix with an affinity chromatography support;
washing the affinity chromatography support with a wash buffer;
collecting a flow-through;
performing fractionation on a sample obtained after performing the enrichment step; and
characterizing at least one of the host-cell proteins using a mass spectrometer.

24. The method of claim 23, wherein the flow-through has a reduced amount of protein of interest than the sample matrix.

25. A method for characterizing host-cell proteins in a sample matrix, comprising:
enriching host-cell proteins in said mixture by contacting the sample matrix with an affinity chromatography support to obtain a mixture;
subjecting the mixture to non-denaturing digestion conditions; and
characterizing at least one of the host-cell proteins using a mass spectrometer.

26. The method of claim 25, wherein the affinity chromatography support is protein A chromatography support.

27. The method of claim 25 further comprising collecting the flow-through from the affinity chromatography support.

28. The method of claim 25, wherein the affinity chromatography support comprises protein A or protein G.

29. The method of claim 28, wherein the protein A or the protein G is immobilized on agarose or sepharose resin.

30. The method of claim 25, wherein the mass spectrometer is a tandem mass spectrometer.

31. The method of claim 25, wherein the mass spectrometer is coupled with a liquid chromatography system.

32. The method of claim 31, wherein the liquid chromatography system is a nano-liquid chromatography system.

33. The method of claim 25, wherein the mass spectrometer is a High-Field Asymmetric Waveform Ion Mobility Spectrometer.

34. The method of claim 28, wherein the sample matrix further comprises a protein of interest.

35. The method of claim 34, wherein the protein of interest is at least one selected from the group consisting of an antibody or a fragment or derivative thereof, a fusion protein, and a physiologically active non-antibody protein.

36. The method of claim 35, wherein the method is capable of characterizing at least about 500% more host-cell proteins than a method that subjects the mixture to non-denaturing digestion conditions to form a mixture without enriching host-cell proteins in said mixture by contacting the sample matrix with an affinity chromatography support to obtain a mixture.

37. The method of claim 34, wherein the method is capable of characterizing at least about 100% to about 1000% more host-cell proteins than a method subjects the mixture to non-denaturing digestion conditions to form a mixture without enriching host-cell proteins in said mixture by contacting the sample matrix with an affinity chromatography support to obtain a mixture.

38. A method for characterizing host-cell proteins in a sample matrix, comprising:
enriching host-cell proteins in the sample matrix by contacting the sample matrix with an affinity chromatography support; and
characterizing at least one of the host-cell proteins using a High-Field Asymmetric Waveform Ion Mobility Spectrometry.

39. The method of claim 38, wherein the method is capable of characterizing at least about 30% more host-cell proteins than a method not comprising a High-Field Asymmetric Waveform Ion Mobility Spectrometry.

40. The method of claim 38, wherein the method is capable of characterizing at least about 30% to about 75% more host-cell proteins than a than a method not comprising a High-Field Asymmetric Waveform Ion Mobility Spectrometry.

41. A method for characterizing host-cell proteins in a sample matrix, comprising:
enriching the host-cell proteins in the sample matrix by contacting a sample matrix with an affinity chromatography support to obtain a mixture;
subjecting the mixture to non-denaturing digestion conditions; and
characterizing of at least one of the host-cell proteins using a High-Field Asymmetric Waveform Ion Mobility Spectrometry.

42. The method of claim 41, wherein the method is capable of characterizing at least about 15% more host-cell proteins than a method that enriches the host-cell proteins in the sample matrix by contacting a sample matrix with an affinity chromatography support to obtain a mixture and subjects the mixture to non-denaturing digestion conditions and characterizing of at least one of the host-cell proteins using a mass spectrometry device other than a High-Field Asymmetric Waveform Ion Mobility Spectrometry device.

43. The method of claim 41, wherein the method is capable of characterizing at least about 15% to about 60% more host-cell proteins than a method that enriches the host-cell proteins in the sample matrix by contacting a sample matrix with an affinity chromatography support to obtain a mixture and subjects the mixture to non-denaturing digestion conditions and characterizing of at least one of the host-cell proteins using a mass spectrometry device other than a High-Field Asymmetric Waveform Ion Mobility Spectrometry device.
